(19) United States
(12) Patent Application Publication
(10) Pub. No.: US 2024/0182665 A1
(43) Pub. Date: Jun. 6, 2024

(54) METHODS FOR CHEMICAL RECYCLING OF CONDENSATION POLYMERS

(71) Applicant: BOARD OF TRUSTEES OF MICHIGAN STATE UNIVERSITY, East Lansing, MI (US)

(72) Inventors: Muhammad Rabnawaz, East Lansing, MI (US); Ajmir Khan, East Lansing, MI (US); Zahra Aayanifard, Okemos, MI (US); Muhammad Naveed, East Lansing, MI (US)

(21) Appl. No.: 18/285,684

(22) PCT Filed: Apr. 5, 2022
(86) PCT No.: PCT/US22/23444

§ 371 Date: Oct. 5, 2023

Related U.S. Application Data
(60) Provisional application No. 63/171,710, filed on Apr. 7, 2021.

(51) Int. Cl. C08J 11/24; B01J 31/02; B01J 31/22; C07C 29/128; C07C 67/475; C08J 11/14; C08J 11/28

(52) U.S. Cl. CPC C08J 11/24; B01J 31/0237; B01J 31/2226; C07C 29/1285; C07C 67/475; C08J 11/14; C08J 11/28; B01J 2231/005; B01J 2531/26; C08J 2367/02; C08J 2367/04; C08J 2377/06

(57) ABSTRACT

The disclosure relates to a method for chemically recycling a condensation polymer, which includes melt-processing a mixture including a condensation polymer and an internal catalyst to increase the amorphous content of the polymer, followed by depolymerizing polymer in a reaction medium with a reactive solvent. Melt-processing and quenching of a condensation polymer generally reduces the crystalline content of the polymer and correspondingly increases the amorphous content of the polymer, which makes the polymer more amenable to subsequent depolymerization. Inclusion of the internal catalyst, for example a volatile organic catalyst, during melt-processing not only improves the relative degree of amorphization during melt-processing, but it also enhances the rate and conversion of the depolymerization stage that would otherwise be rate-limited by mass transport of an external catalyst from the bulk reaction medium to the polymer surface for depolymerization.

### CROSS REFERENCE TO RELATED APPLICATION

Priority is claimed to U.S. Provisional Application No. 63/171,710 filed on Apr. 7, 2021, which is incorporated herein by reference in its entirety.

### STATEMENT OF GOVERNMENT INTEREST

None.

### BACKGROUND OF THE DISCLOSURE

### Field of the Disclosure

The disclosure relates to a method for chemically recycling a condensation polymer, which includes melt-processing a mixture including a condensation polymer and an internal catalyst to increase the amorphous content of the polymer, followed by depolymerizing the polymer in a reaction medium with a reactive solvent. Inclusion of the internal catalyst during melt-processing not only improves the relative degree of amorphization during melt-processing, but it also enhances the rate and conversion of the depolymerization stage.

### Brief Description of Related Technology

Due to the widespread global use of plastics, recycling of plastics continues to be an important field of research. A plastic recycling process should be efficient, environmentally friendly, and cost-effective. Important aspects for any plastic recycling process include: i) adequate supply of the waste plastic; ii) effective systems for collection, transportation, handling, and pre-treatment of the waste plastic; and iii) economic viability of the recycling process.

Condensation polymers such as polyesters and polyamides are particularly attractive targets for recycling since they can be depolymerized to form their component monomers or derivatives thereof (i.e., chemical recycling). Condensation polymers are so named because they are produced by condensation reactions between di- or multifunctional monomers. For example, a polyester is typically formed by polymerizing a diol and a dicarboxylic acid, generating water as a side product. Similarly, a polyamide is typically formed by polymerizing a diamine and a dicarboxylic acid, generating water as a side product. Depolymerization of condensation polymers is more readily achieved than depolymerization of carbon-carbon backbone polymers such as polyolefins and polyacrylates due the presence of more labile C—O and C—N bonds in the polymer backbone.

Among condensation polymers, poly(ethylene terephthalate) (PET) and poly(ethylene terephthalate glycol) (PET/G) are particularly important targets for recycling due to their substantial widespread production and use. Annual production of PET/G in the U.S. alone exceeds 10 billion pounds, and PET/G is the single most recycled plastic in the U.S. and Europe, accounting for roughly 18% and >30%, respectively, of total recycled plastic in each region. PET and PET/G can be chemically recycled by depolymerization to generate the component monomers or derivatives thereof, which can be useful chemical feedstocks for PET and PET/G production or for other chemical processes. As indicated in Path #3 of FIG. 1, such chemical recycling enables a true “circular” approach to polymer production. However, mechanical recycling of PET is currently cheaper, and accordingly much more prevalent, than chemical recycling of PET. As illustrated in FIG. 1, most recycled PET and PET/G bottles are mechanically recycled (path #1 and path #2), such as by downcycling to fibers for low-end applications (˜80%; path #2) or by blending with virgin PET produced from petrochemical feedstocks to form bottles (˜20%; path #1). Ultimately, mechanically-recycled PET predominantly ends up in landfills or incinerators. The disadvantages of mechanical recycling of r-PET/G include reduction of the polymer's molecular weight (Mw) and viscosity due to polymer chain scissions during the melt-processing, which weakens the mechanical properties of the r-PET/G. These losses in performance are often compensated for by the addition of virgin plastics (30-70%) as well as the use of energy intensive solid-stating technique and chain extension methods. In addition, mechanical recycling generally includes impurities found in r-PET/G, which restrict their use only to low end and non-food contact applications. Another disadvantage is that the mechanical recycling is a linear plastic economy approach as mechanically recycled r-PET/G products (e.g., fibers and bottles) ends up in landfills/incinerators, while virgin PET/G resins are prepared from petrochemicals-derived monomers to meet the market need. Thus, this primarily “linear” approach does little to reduce the environmental impact of PET and PET/G production.

Chemical recycling of PET remains less economically viable than mechanical recycling. PET depolymerization processes, and purification of the resulting monomer-rich feedstocks, are time- and energy-intensive; accordingly, petrochemical-derived monomers for PET and PET/G production are generally lower in cost compared to monomers derived from PET depolymerization. To achieve a sustainable, circular process for PET production as suggested in FIG. 1, economically viable methods for chemical recycling of r-PET/G are needed to address these challenges. Previous studies related to chemical recycling and depolymerization of PET/G have focused on identifying effective catalysts, optimizing high-temperature treatment conditions, and using organic solvents to accelerate PET/G depolymerization. However, the current approaches used for the chemical recycling of r-PET/G yield feedstock monomers that are more expensive than those prepared directly from petrochemicals, thus making the chemical recycling a cost-prohibitive process. Multiple factors contribute to the costs associated with the chemical recycling including but not limited to energy-intensive depolymerization performed at higher temperatures and lengthier time as well as complicated purification processes due to mixed chemical composition of r-PET/G in recycled PET along with additives found in recycled PET/G. The low cost of petrochemicals derived r-PET/G monomers puts significant pressure on r-PET/G. As a result and as noted above, r-PET/G is generally mechanically recycled, predominantly down-cycled to fibers, and then sent to landfills/incinerators.

### SUMMARY

In one aspect, the disclosure relates to a method for chemically recycling a condensation polymer, the method comprising: melt-processing a mixture comprising a condensation polymer and a catalyst, thereby forming an amorphous feed material comprising an amorphized condensation polymer and the catalyst, wherein the amorphous feed material (or amorphized condensation polymer) has a crystalline polymer content of 30 wt. % or less; and depolymerizing the amorphous feed material in a reaction medium comprising a reactive solvent, thereby forming a product mixture comprising monomers corresponding to the amorphized condensation polymer. The amorphous feed material can be in the form of the amorphized condensation polymer as a continuous matrix with the catalyst substantially well mixed in or otherwise homogeneously distributed throughout the matrix. The original condensation polymer is generally a crystalline/semicrystalline polymer or polymer blend, for example having a crystalline content from 1-100 wt. % (e.g., with the balance being amorphous content). The catalyst generally has condensation activity, for example catalyzing a condensation reaction to form a bond between components (e.g., monomers or polymer chains) while producing a water byproduct and/or catalyzing an exchange reaction between components (e.g., monomers or polymer chains) to break and re-form condensation bonds. For example, for a polyester polymer, the catalyst can have one or both of condensation activity and transesterification activity. Similarly, for a polyamide polymer, the catalyst can have one or both of condensation activity and transamidation activity.

Melt-processing and quenching of a condensation polymer generally reduces the crystalline content of the polymer and correspondingly increases the amorphous content of the polymer. As illustrated in FIG. 2, increased amorphous content in turn makes the polymer more amenable to subsequent depolymerization, which leads to a more efficient conversion of the condensation polymer and yield of its corresponding monomers as desired depolymerization reaction products. The inclusion of the catalyst at the melt-processing stage improves both the amorphization of the initial condensation polymer as well as the subsequent depolymerization of the polymer. During melt-processing, the catalyst can promote transesterification reactions (e.g., for a polyester condensation polymer), which can further reduce crystallinity beyond the heating/melting effect, in particular when the melt-processed mixture further includes a monomer additive complementary to/reactive with the condensation polymer (e.g., cyclohexane dimethanol (CHDM) or other diol for a polyester condensation polymer). In addition to its effect during melt-processing, the catalyst remains in the amorphized condensation polymer, which in turn enhances the rate and conversion of the depolymerization stage. For example, while the reactive solvent can be efficiently absorbed by and penetrate into solid phase amorphized condensation polymer in the reaction medium, similar transport of catalyst from the bulk reaction medium into the polymer is generally slow and rate-limiting for depolymerization. By incorporating the catalyst into the polymer during melt-processing (or “internal catalyst”), reactive solvent penetrating into the solid polymer can react via catalysis from the internal catalyst without being rate-limited by the uptake of catalyst from the bulk reaction medium (or “external catalyst”).

Inclusion of the (internal) catalyst in the melt-processed mixture can improve amorphization even when the melt-processed mixture does not include a monomer additive complementary to/reactive with the condensation polymer (e.g., CHDM or otherwise). For example, the inclusion of some small amount of moisture or water during melt-processing can increase the amorphous content by partially hydrolyzing the starting condensation polymer thereby reducing the overall molecular weight and increasing the relative number of polymer chain ends, which in turn increases the free volume within the polymer and reduces the overall crystallinity. For example, if the original condensation polymer feed consists only of PET, the presence of the catalyst along with some water can improve amorphization beyond the effect obtained by simply melting in the absence of the catalyst. Similarly, if the original condensation polymer feed consists of PET and PETG, then the presence of the catalyst can have a transesterification randomization effect to redistribute the CHDM units present in the original PETG to the original PET (e.g., forming a new polymer (e.g., “PTEG*”) that has a reduced amount of CHDM units compared to the original PETG). This randomization and redistribution effect reduces the overall crystallinity of the original blend (e.g., the original crystallinity of a PET/PETG blend). In various embodiments, the melt-processed mixture can include 0.0001 wt. % to 1 wt. % water relative to the melt-processed mixture as a whole or the condensation polymer portion thereof.

The monomers corresponding to the amorphized condensation polymer generally include reaction products between the repeat units of the amorphized condensation polymer and the reactive solvent. For example, water can be selected as the reactive solvent for depolymerization via hydrolysis, forming diacid and diol monomers (for a polyester condensation polymer) or diacid and diamine monomers (for a polyamide condensation polymer). Similarly, a mono-alcohol (e.g., methanol) can be selected as the reactive solvent for depolymerization via alkylolysis (e.g., methanolysis), forming diester and diol monomers (for a polyester condensation polymer) or diester and diamine monomers (for a polyamide condensation polymer). In addition to the resulting monomers, the product mixture generally contains any reactive solvent not consumed during depolymerization as liquid medium containing the formed monomers therein, along with remaining catalyst (internal and/or external), other reaction medium components, etc.

In another aspect, the disclosure relates to a method for chemically recycling a condensation polymer, the method comprising: melt-processing a mixture comprising a condensation polymer and, optionally, an internal catalyst, thereby forming an amorphous feed material comprising an amorphized condensation polymer and the internal catalyst (when present), wherein the amorphous feed material has a crystalline polymer content of 30 wt. % or less; and depolymerizing the amorphous feed material in a reaction medium comprising (i) a reactive solvent and (ii) an external catalyst comprising an organic base, thereby forming a product mixture comprising monomers corresponding to the amorphized condensation polymer. The internal catalyst, when included in the melt-processed mixture, can include any of the catalysts described herein, whether metal-based catalysts, organic catalyst (e.g., amidine base), or otherwise. In some embodiments, the melt-processed mixture does not contain or is otherwise free from the internal catalyst generally and/or an organic base more specifically. In some embodiments, the melt-processed mixture contains the internal catalyst, for example where the internal catalyst is other than an organic base (e.g., the melt-processed mixture is free from organic base internal catalysts but includes a metal-based or other internal catalyst). In some embodiments, the organic base external catalyst comprises a volatile organic base catalyst.

Various refinements of the disclosed methods are possible.

In a refinement, the condensation polymer is selected from the group consisting of polyesters, polyamides, and combinations thereof. The condensation polymers are generally thermoplastic polymers such as thermoplastic polyesters and polyamides. The melt-processed mixture can include a single condensation polymer along with a catalyst, or it can include two or more different condensation polymers along with the catalyst. For example, initial melt-processed mixture can include two or more polyesters, two or more polyamides, at least one polyester and at least one polyamide, etc. Examples of suitable polyamides include nylon 6,6, nylon 6, nylon 6,10, etc. Examples of other suitable thermoplastic polymers that can be melt-processed and amorphized according to the disclosure include polycarbonates, polyanhydrides, polyimides, polybenzimidazoles, polyquinoxlines, armoatic ladder polymers, phenol-formaldehyde polymers, urea-formaldehyde polymers, melamine-formaldehyde polymers, polyacetals, polyethersulfones, polyethers, polyphenylene oxides, polyarylenes, and thermoplastic polyurethanes.

In a refinement, the condensation polymer comprises at least one polyester selected from the group consisting of polyethylene terephthalate (PET), polyethylene terephthalate glycol-modified (PETG; including typically about 5-50 mol. % or 15-30 mol. % cyclohexane dimethanol comonomer with 50-95 mol. % or 70-85 mol. % ethylene glycol comonomer) polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polybutylene succinate terephthalate (PBST), polyethylene succinate (PES), poly(butylene succinate/terephthalate/isophthalate)-co-(lactate) (PBSTIL), liquid crystalline polyesters, and combinations thereof.

In a refinement, the condensation polymer comprises polyethylene terephthalate (PET) and polyethylene terephthalate glycol-modified (PETG). More generally, the condensation polymer can include a blend of condensation polymers, for example PET in combination with one or more copolyesters (e.g., amorphous copolyesters), whether PETG or otherwise. Examples of other copolyesters include isophthalate-modified copolyesters, sebacic acid-modified copolyesters, diethyleneglycol-modified copolyesters, triethyleneglycol modified-copolyesters, cyclohexanedimethanol modified-copolyesters, and/or polybutylene terephthalate. Such modified copolyesters generally have at least one of the TPA or EG units in PET at least partially replaced with modifying unit (e.g., at least some terephthalic units replaced with isophthalic units, at least some ethylene glycol units replaced with diethyleneglycol units), for example with 2-50 mol. %, 5-50 mol. %, 10-40 mol. %, 10-20 mol. %, 20-30 mol. %, or 15-30 mol. % replacement by the modifying unit. In some embodiments, the copolyesters can have a low melting point or include an amorphous aromatic copolyester (such as one based on terephthalate/isophthalate copolymer with ethylene glycol or a copolyester made from a combination of terephthalic acid, ethylene glycol, and cyclohexyldimethanol). In various embodiments, the condensation polymer can include 5-95 wt. % PET (e.g., at least 5, 10, 15, 20, 30, 40, 50, 60, 70, or 80 wt. % and/or up to 20, 30, 40, 50, 60, 70, 80, 90, or 95 wt. %) relative to the total amount of condensation polymers in the initial melt-processed mixture. In various embodiments, the condensation polymer can include 5-95 wt. % PETG or other copolyesters (e.g., at least 5, 10, 15, 20, 30, 40, 50, 60, 70, or 80 wt. % and/or up to 20, 30, 40, 50, 60, 70, 80, 90, or 95 wt. %) relative to the total amount of condensation polymers in the initial melt-processed mixture. In some embodiments, the disclosed method and melt-processing can be performed on combinations of polyesters or other condensation polymers in multilayer materials, for example laminated polyester structures (e.g., where different layers can have different polyester components or compositions).

In a further refinement, the condensation polymer further comprises polylactic acid (PLA) (i.e., in addition to PET and PETG or other copolyester(s)). In various embodiments, the condensation polymer can include 0.01-10 wt. % PLA (e.g., at least 0.01, 0.1, 1, 2, 3, or 5 wt. % and/or up to 2, 3, 4, 6, 8, or 10 wt. %) relative to the total amount of condensation polymers in the initial melt-processed mixture.

In a refinement, the catalyst is selected from the group consisting of metal alkanoates, metal benzoates, metal carbonates, metal sulfates, acids, bases, and combinations thereof. Suitable metals in the various catalysts can include zinc, zirconium, titanium, sodium, potassium, etc. Example metal alkanoates include zinc acetate, and zinc ethylhexanoate, and zinc fluoroacetate. Example metal benzoates include zinc benzoate, zinc chlorobenzoate, zinc bromobenzoate, zinc nitrobenzoate, and zinc methoxy benzoate. Example metal sulfates include sodium sulfate and potassium sulfate. Example metal carbonates include sodium carbonate and potassium carbonate. Example acid catalysts include sulfuric acid and sulfonic acid derivatives. Example bases include metal hydroxides such as sodium hydroxide and potassium hydroxide. The foregoing catalysts can be used as condensation catalysts for the condensation polymers described herein, for example also serving as a transesterification catalyst (e.g., when the condensation polymer includes a polyester) and/or a transamidation catalyst (e.g., when the condensation polymer includes a polyamide).

In a refinement, the catalyst can be an organic base, for example selected from organic compounds which are metal-free or substantially metal-free. Suitable organic catalysts include 1,5,7-triazabicyclo[4.4.0]dec-5-ene (TBD) and 1,8-diazabicyclo[5.4.0]undec-7-ene (DBU). The foregoing catalysts can be used as condensation catalysts for the condensation polymers described herein, for example also serving as a transesterification catalyst (e.g., when the condensation polymer includes a polyester) and/or a transamidation catalyst (e.g., when the condensation polymer includes a polyamide). Furthermore, the foregoing catalysts can be substantially removed from the depolymerized reaction mixture via distillation, obviating the need for complex and energy-intensive filtration operations. For example, the organic base can have a boiling point of at least 80, 100, 120, 150, or 200° C. and/or up to 100, 120, 150, 170, 200, 250, or 300° C. such as a relatively volatile organic base. Thus, in a further refinement, the method further comprises: recovering the organic base from the product mixture; and optionally performing a subsequent melt-processing of a new condensation polymer with the recovered organic base as the catalyst.

In a refinement, the catalyst is present in the melt-processed mixture in an amount in a range from 0.1 wt. % to 20 wt. % or 1 wt. % to 5 wt. % relative to the melt-processed mixture. More generally, the catalyst can be present in amount of at least 0.1, 0.2, 0.5, 1, or 2 wt. % and/or up to 2, 4, 5, 6, 8, 10, 12, 15, or 20 wt. % relative to the melt-processed mixture. The catalyst added to the melt-processed mixture remains after melt-processing, so it is predominantly present in the amorphous feed material in corresponding amounts relative to the amorphous feed material. The foregoing ranges are expressed relative to the mixtures including the total amount of (amorphized) condensation polymer(s) and catalyst. Alternatively or additionally, the foregoing ranges can be expressed relative to the total amount of (amorphized) condensation polymer(s) only (e.g., excluding catalyst weight and any other additive such as comonomers).

In a refinement, the catalyst is heat-stable at temperatures experienced during the melt-processing. The catalyst added to the mixture for melt-processing is suitably heat-stable or heat-resistant so that it does not degrade or otherwise lose its activity during melt-processing. This permits the catalyst to remain in the amorphous feed material as an active internal catalyst that also can catalyze depolymerization in the next step. For example, after exposure to the high melting temperatures during melt processing, the catalyst suitably is present in amounts of at least 20, 50, 85, 90, 95, or 98 wt. % and/or up to 90, 95, 99, or 100 wt. % relative to its initial amount (e.g., representing that substantial amounts of the catalyst do not decompose or degrade to other components upon heating). Alternatively or additionally, after exposure to the high melting temperatures during melt processing, the catalyst suitably retains at least 20, 50, 80, 85, 90, 95, or 98% and/or up to 90, 95, 99, or 100% of its initial catalytic activity (e.g., representing that substantial amounts of the catalyst are not inactivated upon heating). As a class of catalysts, metal benzoates (e.g., substituted or unsubstituted benzoates such as zinc benzoates) are particularly heat-stable and useful at higher melt-processing temperatures according to the disclosure (e.g., 280° C. and higher), for example relative to zinc acetate.

In a refinement, the melt-processed mixture further comprises a monomer reactive with the condensation polymer. The added monomers generally include compounds that are complementary to/reactive with repeat units of the condensation polymer. For example, the added monomers can include diols (or polyols) or diacids (or polyacids) for addition to polyester condensation polymers (i.e., reactive with the diacid or diol units thereof, respectively), diamines (or polyamines) or diacids (or polyacids) for addition to polyamide condensation polymers (i.e., reactive with the diacid or diamine units thereof, respectively), etc. The added monomers are incorporated into the condensation polymers via transesterification, transamidation, etc. that is catalyzed by the catalyst in the mixture at the high melt-processing temperature. The added monomers can be selected to reduce the crystallinity of the amorphized condensation polymer in which they are incorporated, impart some additional functionality to the amorphized condensation polymer, etc. Examples include diethylene glycol (DEG), 1,4-cyclohexane dimethanol (CHDM), bis(2-hydroxyethyl) terephthalate (BHET). Monomers such as BHET is added to provide more hydroxyl groups and reduce the PET structure (e.g., via chain scission during transesterification).

More generally, suitable diols can be represented by HO—R—OH (where R includes alkyl, cyclic, and/or aryl groups). The R group can have 2-40 carbon atoms (e.g., at least 2, 4, 6, 8 or 10 and/or up to 4, 8, 12, 20, 30, or 40), and the R can further include one or more heteroatoms such as O, N, S, etc. Specific examples of polyols include glycerol, ethylene glycol, diethylene glycol, triethylene glycol, tetraethylene glycol, propylene glycol, dipropylene glycol, tripropylene glycol, 1,3-propanediol, 1,3-butanediol, 1,4-butanediol, neopentyl glycol, 1,6-hexanediol, 1,8-octanediol, 1,4-cyclohexanedimethanol, glycerol, trimethylolpropane, 1,2,6-hexanetriol, pentaerythritol, (meth)acrylic polyols, isosorbide, and combinations thereof. Higher-functional polyols such as triols etc. can be useful to reduce crystallinity.

More generally, suitable diacids can be represented by HOOC—R—COOH (where R includes alkyl, cyclic, and/or aryl groups). The R group can have 2-40 carbon atoms (e.g., at least 2, 4, 6, 8 or 10 and/or up to 4, 8, 12, 20, 30, or 40), and the R can further include one or more heteroatoms such as O, N, S, etc. Specific examples of dicarboxylic acids or polyacids include adipic acid, sebacic acid, isophthalic acid, phthalic acid, 2,6-naphthalenedicarboxylic acid, 4,4′-biphenyldicarboxylic acid, 4,4′-oxybisbenzoic acid, 5-sulfoisophthalic acid monosodium salt, 1,4-butanedioic acid, 1,6-hexanedioic acid, decanedioic acid, 1,4-cyclohexanedicarboxylic acid, etc.

More generally, suitable diamines can be represented by H₂N—R—NH₂ (where R includes alkyl, cyclic, and/or aryl groups). The R group can have 2-40 carbon atoms (e.g., at least 2, 4, 6, 8 or 10 and/or up to 4, 8, 12, 20, 30, or 40), and the R can further include one or more heteroatoms such as O, N, S, etc. Specific examples of diamines or polyamines include 1,6-hexanediamine, ethylene diamine, toluene diamine, diaminodiphenylmethane and polymethylene polyphenylene polyamines, aminoalcohols, etc. The amino functional group can be primary, secondary, or tertiary. A specific example of a suitable polyetheramine is polyoxypropylenediamine (e.g., JEFFAMINE D-400 as a diamine). Higher-functional polyamines such as triamines etc. as well as a branched polyamines can be useful to reduce crystallinity.

In a further refinement, the monomer is present in the melt-processed mixture in an amount in a range from 0.5 wt. % to 90 wt. %, 5 wt. % to 30 wt. %, or 0.5 wt. % to 5 wt. % or relative to the melt-processed mixture. In various embodiments, the monomer can be present in the melt-processed mixture in amount of at least 0.5, 1, 2, 5, 8, 10, 15, 20, or 30 wt. % and/or up to 5, 10, 15, 20, 25, 30, 50, 70, or 90 wt. % relative to the initial melt-processed mixture. The monomer added to the melt-processed mixture is generally incorporated into the amorphous condensation polymer as a repeat unit therein via catalyzed reaction with the initial condensation polymer, although some monomers can remain unreacted, and/or some monomers can remain as chain end-one end inserted units. The foregoing ranges are expressed relative to the mixtures including the total amount of condensation polymer(s), catalyst, and monomers. In various embodiments, the combined amount of condensation polymer(s), catalyst, and monomers (when present) corresponds to at least 95, 98, 99, or 99.5 wt. % and/or up to 97, 98, 99, 99.5, 99.8, or 100 wt. % of the (initial) melt-processed mixture. Suitably, there are no added reactive solvents (e.g., water, methanol, or other mono-alcohols) in the (initial) melt-processed mixture, for example containing not more than 2, 1, 0.5, 0.2, or 0.1 wt. % of such reactive solvents in the mixture. In some embodiments, however, the melt-processed mixture can include 0.0001 wt. % to 1 wt. % water to promote transesterification, which small amounts of water can be due to naturally present or ambient moisture (e.g., no added water) or (alternatively) some amount of added water. For example, the (initial) melt-processed mixture can include at least 0.0001, 0.001, 0.01, 0.1, or 0.2 wt. % and/or up to 0.1, 0.2, 0.4, 0.6, 0.8, or 1 wt. % water relative to the melt-processed mixture as a whole or the condensation polymer portion thereof. In embodiments where some water is present in the melt-processed mixture, the mixture is suitably free or substantially free of other reactive solvents in general and free or substantially free of added water (i.e., non-ambient or non-environmental water).

In a refinement, the condensation polymer in the melt-processing mixture (e.g., as initially present therein or added thereto) has a crystalline polymer content of 20 wt. % or more; and/or the amorphized condensation polymer in the amorphous feed material (e.g., after melt-processing and/or quenching, but before depolymerization) has a crystalline polymer content of 15 wt. % or less. More generally, the condensation polymer in the initial melt-processing mixture can have a crystalline polymer content of at least 5, 10, 20, 25, 30, 35, 40, 45, 50, 60, 70, or 80 wt. % and/or up to 10, 20, 30, 40, 50, 60, 70, 80, 90, or 100 wt. %. Alternatively or additionally, the condensation polymer in the initial melt-processing mixture can have an amorphous polymer content of at least 1, 2, 5, 10, 20, 30, 40, 50, 60, 70, 80, or 90 wt. % and/or up to 20, 30, 40, 50, 60, 70, 80, 90, 95, or 98 wt. %. Alternatively or additionally, the amorphous condensation polymer in the amorphous feed material can have a crystalline polymer content of at least 0.1, 0.2, 0.5, 1, 2, 4, 6, 8, 10, 12, 15, or 20 wt. % and/or up to 1, 2, 3, 5, 7, 10, 15, 20, 25, or 30 wt. %. Alternatively or additionally, the amorphous condensation polymer in the amorphous feed material can have an amorphous polymer content of at least 60, 65, 70, 75, 80, 85, 90, 95, 98, or 99 wt. % and/or up to 70, 75, 80, 90, 95, 98, 99, 100 wt. %. Alternatively or additionally, a difference between the crystalline polymer content of the condensation polymer in the initial melt-processing mixture and the amorphous feed material can be at least 1, 2, 5, 10, 15, 20, or 30 wt. % and/or up to 5, 10, 20, 30, 40, 50, 60, 70, or 80 wt. % (i.e., reflecting a reduction in crystalline content). Alternatively or additionally, a difference between the amorphous polymer content of the condensation polymer in the amorphous feed material and the initial melt-processing mixture can be at least 1, 2, 5, 10, 15, 20, or 30 wt. % and/or up to 5, 10, 20, 30, 40, 50, 60, 70, or 80 wt. % (i.e., reflecting an increase in amorphous content). The foregoing ranges for crystalline and amorphous polymer content can apply to single condensation polymers in the mixtures being processed and/or collectively to all condensation polymers in the mixtures being processed (e.g., average crystalline and/or amorphous polymer contents for the processed blends).

In a refinement, melt-processing comprises heating the mixture to a temperature in a range of 260° C. to 320° C. Any apparatus typically used for melt-processing of the condensation polymers may be used, for example twin- and single-screw extrusion apparatus operated at a sufficiently high temperature to melt the base condensation polymer material. Temperatures in a range of 100° C. to 400° C., 200° C. to 300° C., 260° C. to 320° C., or 280° C. to 300° C., for example at least 100, 150, 170, 200, 220, 250, 280, or 300° C. and/or up to 160, 180, 210, 240, 280, 300, 320, 350, or 400° C., are particularly suitable for polyester condensation polymers such as PET and/or PETG. Alternatively or additionally, the melt-processing temperature can be expressed relative to the highest melting temperature (Tm) for the condensation polymer(s) in the melt-processed mixture. For example, if the melt-processed mixture includes two polymers, one with a melting point of 260° C. and another with a melting point of 280° C., then the highest melting temperature (Tm) is 280° C. In some embodiments can melt-processing temperature can be at least 5, 10, 15, 20, 25, 40, 60, or 100° C. above the highest melting temperature (Tm) and/or up to 10, 15, 25, 35, 50, 65, 80 or 100° C. above the highest melting temperature (Tm).

In a refinement, the method further comprises: quenching the melt-processed amorphous feed material in a liquid medium prior to depolymerizing. The quenching in a (relatively) cold liquid medium after extrusion or other melt-processing can be used to form filaments or strands of the amorphous feed material to limit or prevent crystal growth therein prior to depolymerization. The liquid medium for quenching can be the same material(s) used for the reaction medium. A suitable quenching temperature is below the glass transition temperature (Tg) of the melt-processed polymer or polymer blend, for example at least 5, 10, 15, or 20° C. below Tg and/or in a range of 0-60° C., 10-50° C., or 20-40° C. Alternatively or additionally, the quenching temperature can be below the crystallization temperature (Tc) of the melt-processed polymer or polymer blend, for example at least 5, 10, 15, or 20° C. below Tc.

In a refinement, the reactive solvent is selected from the group consisting of water, mono-alcohols (e.g., methanol, ethanol), diols, mono-amines, diamines, and combinations thereof. In addition to mono-alcohols, diols or polyols such as ethylene glycol, glycerols, and those mentioned above can be used. Suitable amino compounds can include mono-amines, diamines, polyamines, ammonia, and alcoholic amines (e.g., an amino alkanol). In various embodiments, the reaction medium and/or the reaction solvent portion thereof can be present in a relative amount of at least 2, 5, 7, 10, 12, 15, or 20 and/or up to 6, 8, 10, 15, 20, 30, 40, or 50 on a w/w or v/v basis relative to the amorphous feed material. In some embodiments, the reaction medium can optionally include non-reactive solvents such as organic non-alcohol or non-amine solvents (e.g., non-protic or aprotic solvents such as tetrahydrofuran, chloroform, etc.) to speed up the depolymerization by enhancing the swelling the of the amorphous polymer (e.g., PET/G). In addition to increasing the rate of depolymerization (e.g., via polymer swelling), the addition of non-reactive solvents can be used to enable phase-separation and recovery of the reaction products (e.g., using fluorinated organic solvents, ionic liquids, etc.). When included, the non-reactive solvent can similarly be included in a relative amount of at least 2, 5, 7, 10, 12, 15, or 20 and/or up to 6, 8, 10, 15, 20, 30, 40, or 50 on a w/w or v/v basis relative to the amorphous feed material.

In a refinement, the reactive solvent comprises water (e.g., for depolymerization via hydrolysis); the condensation polymer comprises a polyester (e.g., likewise for the amorphized condensation polymer); and the monomers in the product mixture comprise diacids and diols formed from the repeat units of the polyester. For example, if the original condensation polymer is PET depolymerized via hydrolysis, then the corresponding monomers include terephthalic acid (TPA) and ethylene glycol (EG). If a monomer is added to the melt-processed mixture, then such monomer can also be recovered in the product mixture. For example, if the original condensation polymer is PET melt-processed with CHDM and then depolymerized via hydrolysis, then the corresponding monomers include TPA, EG, and CHDM.

In a refinement, the reactive solvent comprises methanol (e.g., for depolymerization via methanolysis); the condensation polymer comprises a polyester (e.g., likewise for the amorphized condensation polymer); and the monomers in the product mixture comprise diesters and diols formed from the repeat units of the polyester. For example, if the original condensation polymer is PET depolymerized via methanolysis, then the corresponding monomers include dimethyl terephthalate (DMT) and ethylene glycol (EG). If a monomer is added to the melt-processed mixture, then such monomer can also be recovered in the product mixture. For example, if the original condensation polymer is PET melt-processed with CHDM and then depolymerized via methanolysis, then the corresponding monomers include DMT, EG, and CHDM. Analogous ethyl or other alkyl esters can be formed when using mono-alcohols other than methanol, such as ethanol, etc.

In a refinement, the reactive solvent comprises ethylene glycol (e.g., for depolymerization via glycolysis); the condensation polymer comprises a polyester (e.g., likewise for the amorphized condensation polymer); and the monomers in the product mixture comprise diesters formed from the repeat units of the polyester. For example, if the original condensation polymer is PET depolymerized via glycolysis with ethylene glycol, which itself is a monomer for PET, then the corresponding monomers include bis(hydroxyethyl)terephthalate (BHET) and ethylene glycol. If a monomer is added to the melt-processed mixture, then such monomer can also be recovered in the product mixture. For example, if the original condensation polymer is PET melt-processed with CHDM and then depolymerized via glycolysis, then the corresponding monomers include BHET and CHDM.

In a refinement, the reactive solvent comprises water (e.g., for depolymerization via hydrolysis); the condensation polymer comprises a polyamide (e.g., likewise for the amorphized condensation polymer); and the monomers in the product mixture comprise diacids and diamines formed from the repeat units of the polyamide.

In a refinement, the reactive solvent comprises methanol (e.g., for depolymerization via methanolysis); the condensation polymer comprises a polyamide (e.g., likewise for the amorphized condensation polymer); and the monomers in the product mixture comprise diesters and diamines formed from the repeat units of the polyamide.

In a refinement, the reactive solvent comprises at least one of a mono-amine and a diamine; the condensation polymer comprises a polyamide (e.g., likewise for the amorphized condensation polymer); and the monomers in the product mixture comprise diamides and diamines formed from the repeat units of the polyamide.

In various refinements, the reaction medium can either further comprise an external catalyst or be substantially free from external catalysts. For example, in some cases, it is possible to include sufficient internal catalyst at the time of melt-processing so that depolymerization can process simply by heating the amorphized condensation polymer in reaction medium with reactive solvent but without further catalyst. In general, however, it can be desirable to include an external catalyst in the reaction medium to assist or accelerate depolymerization by directly acting on the exposed external surfaces of the amorphized condensation polymer. The external catalyst can generally be selected from the same options as described for the (internal) catalyst added to the melt-processed mixture and incorporated into the amorphous feed material. In some embodiments, the internal catalyst and the external catalyst are the same catalysts. In some embodiments, the internal catalyst and the external catalyst are different catalysts. In embodiments including an external catalyst, the external catalyst can be present in the reaction medium in an amount in a range from 0.1 wt. % to 10 wt. % or 0.5 wt. % to 5 wt. % relative to the reaction medium, for example in an amount of at least 0.1, 0.2, 0.5, 1, or 2 wt. % and/or up to 1.5, 3, 5, 7, or 10 wt. %. In embodiments where no external catalyst is added to the reaction medium, the reaction medium suitably does not contain more than 0.001, 0.01, or 0.1 wt. % of an external catalyst (e.g., the same catalyst species as included for the internal catalyst and/or all catalyst species combined)

In a refinement, the reaction medium further comprises a surfactant. A surfactant can lower the surface energy of the depolymerization reaction medium to improve polymer wetting and depolymerization efficiency. The surfactant suitably can be included in an amount of 0.1-10 wt. % or 0.5-4 wt. % in the reaction medium. The surfactant is not particularly limited, for example including anionic, cationic, zwitterionic, and non-ionic surfactants. Examples of suitable anionic surfactants include sodium dodecyl sulfate (SDS) and sodium lauryl sulfate (SLS).

In a refinement, the method further comprises: swelling the amorphized condensation polymer with at least one of a gaseous swelling and a non-protic solvent swelling agent (e.g., to enhance depolymerization). High-pressure gases such as carbon dioxide and oxygen can swell condensation polymers (e.g., PET, PETG, and other polyesters), making the amorphous regions of the polymer more accessible during depolymerization, as illustrated in FIG. 3. For example, after melt-processing and prior to depolymerization, the amorphized condensation polymer can be exposed to carbon dioxide and/or oxygen at high pressures and above the Tg of the polymer, for example in a pressurized vessel (e.g., which could be the reaction vessel for depolymerization, but without added reaction solvent/reaction medium). The pressure is then released, the swelled amorphized condensation polymer is then added to or otherwise combined with the reaction solvent/reaction medium, and depolymerization can then be performed. As described above, non-protic or aprotic solvents such as tetrahydrofuran, chloroform, etc. can be added to the reaction medium as an additional or alternative means to swell the amorphized condensation polymer to enhance depolymerization.

In a refinement, the reaction medium further comprises at least one of polystyrene (PS) sulfonic acid beads and a hydroxylated pyridine. The PS sulfonic acid beads can be included as a solid acid external catalyst that can be easily recovered and re-used after deopolymerization. The hydroxylated pyridine can serve as a bifunctional catalyst.

In a refinement, depolymerizing comprises heating the amorphous feed material in the reaction medium to a temperature in a range of 120° C. to 200° C. Any reaction vessel typically used for batch, semi-batch, or continuous reactions may be used. Temperatures such as at least 25° C., 60° C., 120° C., or 150° C. and/or up to 50° C., 75° C., 100° C., 150° C., 170° C., or 200° C. are particularly suitable for polyester condensation polymers such as PET and/or PETG. While depolymerization is often performed at elevated temperatures (e.g., above 100° C.), so catalysts such as sodium amide, sodium hydroxide, etc. can drive the depolymerization reaction at lower temperatures as low as ambient (room) temperatures. The depolymerization reaction is suitably performed for a sufficient time (or residence time in a continuous reactor) to essentially completely depolymerize the (amorphized) condensation polymer into its corresponding monomers, for example for at least 0.01, 0.1, 0.5, 1, 2, 3, or 4 hr and/or up to 2, 4, 6, 8, 10, 12, 18, or 24 hr. In various embodiments, the product mixture contains 20, 10, 5, 2, 1, 0.5, 0.2, or 0.1 wt. % or less of the (amorphized) condensation polymer relative to the initial condensation polymer, for example corresponding to a conversion of at least 80, 90, 95, 98, or 99 wt. % and/or up to 95, 98, 99, or 100 wt. % of the condensation polymer.

In a refinement, the condensation polymer comprises at least one of polyethylene terephthalate (PET) and polyethylene terephthalate glycol-modified (PETG); the reactive solvent comprises methanol; the monomers in the product mixture comprise dimethyl terephthalate (DMT), ethylene glycol (EG), and optionally 1,4-cyclohexane dimethanol (CHDM) (e.g., when PETG is an included condensation polymer and/or when CHDM is an added monomer to the melt-processed mixture); and the method further comprises hydrogenating the DMT to form (further) CHDM. Suitable hydrogenation processes are generally known in the art, for example by reaction with hydrogen gas and a suitable catalyst. For example, hydrogenation of DMT can be performed in two steps to first convert DMT to 1,4-cyclohexane dimethylcarboxylate (CHDC), and then convert CHDC to CHDM.

While the disclosed articles, apparatus, methods, and compositions are susceptible of embodiments in various forms, specific embodiments of the disclosure are illustrated (and will hereafter be described) with the understanding that the disclosure is intended to be illustrative, and is not intended to limit the claims to the specific embodiments described and illustrated herein.

### BRIEF DESCRIPTION OF THE DRAWINGS

FIG. 1 illustrates mechanical and chemical recycling of PET, showing a circular pathway of reuse and recycling for chemically-recycled PET vs. a linear pathway for mechanically-recycled PET.

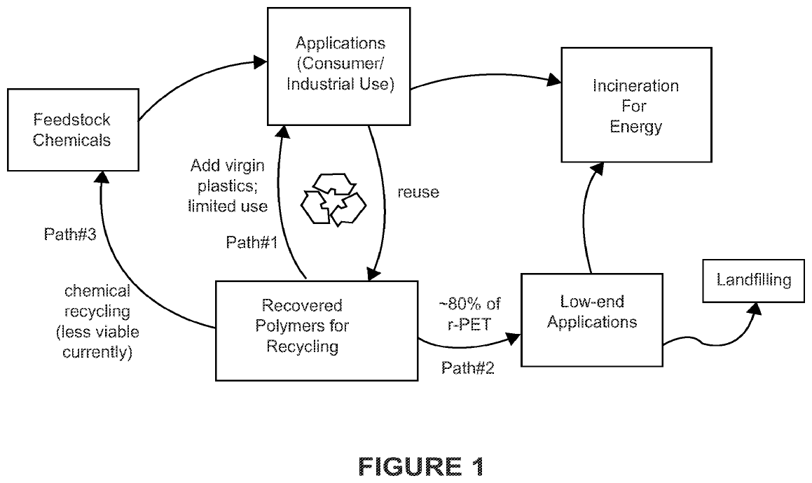

FIG. 2 illustrates a comparison of r-PET/G without pre-treatment (i.e., non-melt-processed PET), which retains some crystalline domains that are poorly accessible during subsequent reactions, and r-PET/G after pre-treatment (i.e., melt-processed PET), which is amorphized and more easily accessible during subsequent reactions.

FIG. 3 shows a schematic comparison between unswollen PET and swollen PET. Unswollen PET has low internal free volume, hindering mass transfer of reagents and products in and out of the polymer. Swollen PET has high internal free volume, facilitating mass transfer of reagents and products in and out of the polymer.

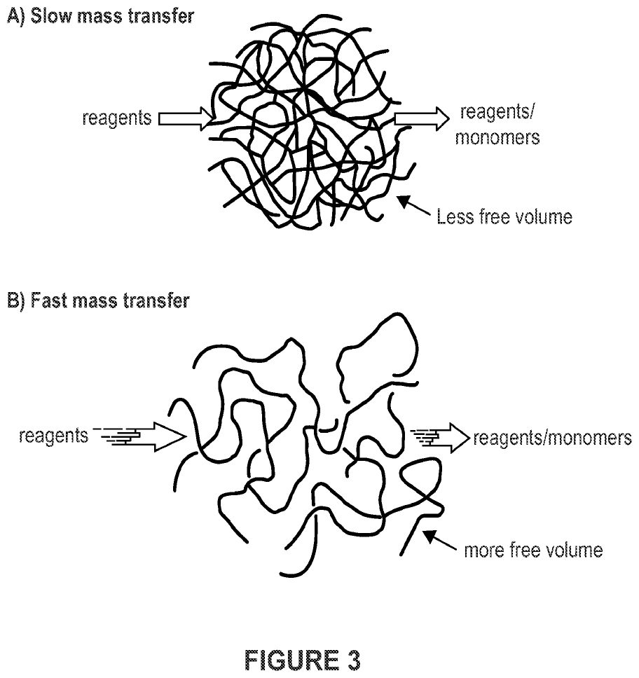

FIG. 4 shows products of methanolysis of r-PET/G and value-added feedstocks that can be generated by r-PET/G methanolysis.

FIG. 5 illustrates that depolymerization nano-sites within r-PET/G can accelerate depolymerization by providing a reaction pathway that is not hindered by diffusion of reagents (i.e., solvent and catalyst) into the polymer.

FIG. 6 shows typical design of an extruder that can be used to melt-process (i.e., amorphize and extrude) r-PET/G or other condensation polymers.

### DETAILED DESCRIPTION

The disclosure relates to a method for chemically recycling a condensation polymer, which includes melt-processing a mixture including a condensation polymer and an internal catalyst to increase the amorphous content of the polymer, followed by depolymerizing the polymer in a reaction medium with a reactive solvent. Melt-processing and quenching of a condensation polymer generally reduces the crystalline content of the polymer and correspondingly increases the amorphous content of the polymer, which makes the polymer more amenable to subsequent depolymerization. Inclusion of an internal catalyst (e.g., metal-based catalyst, organic base catalyst, etc.) during melt-processing improves the relative degree of amorphization during melt-processing and enhances the rate and conversion of the depolymerization stage. Without an internal catalyst, the depolymerization stage can otherwise be rate-limited by mass transport of an external catalyst from the bulk reaction medium to the polymer surface for depolymerization. In some embodiments, however, the chemical recycling method can instead incorporate an organic base as an external catalyst during depolymerization, either with or without an internal catalyst being present during melt-processing.

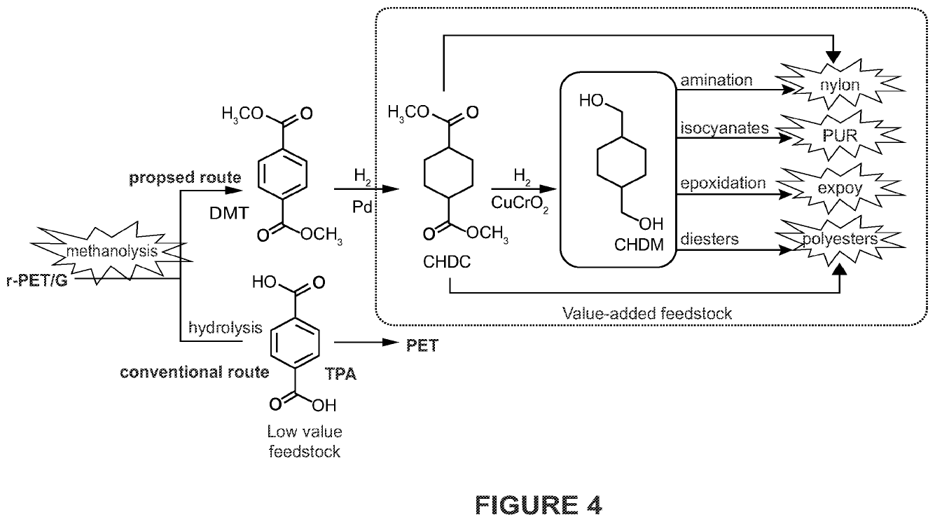

The disclosure relates to recycling of plastics, particularly condensation polymers such as polyesters and polyamides, by chemical rather than mechanical means. Such chemical recycling processes can provide sustainable routes to useful materials. In particular, as shown in FIG. 4, PET/G can be chemically recycled to provide value-added feedstocks which can be used in a wide range of downstream processes. Chemical recycling of PET/G by a conventional hydrolysis route generates terephthalic acid, which can be used to produce PET, while chemical recycling of PET/G by methanolysis can yield higher value feedstocks. In particular, methanolysis of PET/G generates dimethylterephthalate (DMT), which can be reduced to cyclohexane dicarboxylate (CHDC) (e.g., a dimethyl ester as illustrated) and/or cyclohexane dimethanol (CHDM), both of which are useful starting materials for further reactions to form useful materials including nylons, polyurethanes, epoxies, and polyesters. Glycolysis of PET/G generates hydroxylated species that are also useful starting materials for polymerization reactions, including condensation reactions. For example, glycolysis of PET/G using ethylene glycol generates bis(hydroxyethyl)terephthate (BHET), which can be used as a monomer to produce PET.

In particular, the disclosure relates to a method for chemically recycling a condensation polymer by depolymerizing the condensation polymer to form a product mixture including monomers corresponding to repeat units of the condensation polymer. For example, r-PET/G can be depolymerized into monomer product mixtures including predominantly terephthalic acid and ethylene glycol (via hydrolysis) or dimethylterephthalate and ethylene glycol (via methanolysis). The method includes melt-processing a mixture including a condensation polymer and a catalyst to form an amorphous feed material in which the crystalline polymer content of the condensation polymer is no greater than 30%. The amorphous feed material is reacted in a reaction medium including a reactive solvent to effect depolymerization of the condensation polymer.

The amorphous feed material can be in the form of the amorphized condensation polymer as a continuous matrix with the catalyst substantially well mixed in or otherwise homogeneously distributed throughout the matrix. The original condensation polymer is generally a crystalline/semicrystalline polymer or polymer blend, for example having a crystalline content from 1-100 wt. % (e.g., with the balance being amorphous content). The catalyst generally has condensation activity, for example catalyzing a condensation reaction to form a bond between components (e.g., monomers or polymer chains) while producing a water byproduct and/or catalyzing an exchange reaction between components (e.g., monomers or polymer chains) to break and re-form condensation bonds. For example, for a polyester polymer, the catalyst can have one or both of condensation activity and transesterification activity. Similarly, for a polyamide polymer, the catalyst can have one or both of condensation activity and transamidation activity.

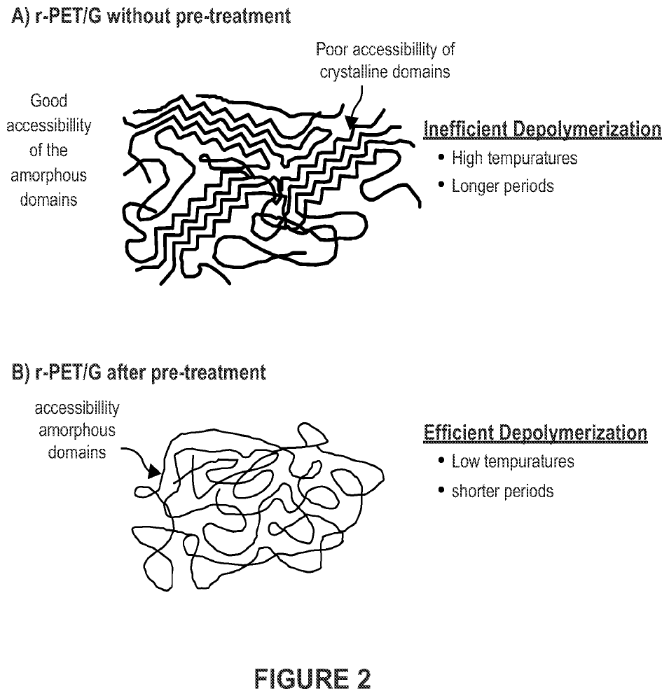

Melt-processing and quenching of a condensation polymer generally reduce the crystalline content of the polymer and correspondingly increase the amorphous content of the polymer. Increased amorphous content in turn makes the polymer more amenable to subsequent depolymerization. The effect of melt-processing on downstream depolymerization is shown schematically in FIG. 2. Native r-PET/G (and other condensation polymers more generally) includes a mix of amorphous, semi-crystalline, and crystalline polymers. However, only the amorphous regions of a polymer allow sufficient mass transfer (e.g., sorption, permeation, and/or diffusion) of other compounds, while crystalline and semi-crystalline regions are generally regarded as impermeable, which in turn makes the crystalline and semi-crystalline regions very difficult to depolymerize due to the poor or lack of accessibility of reagents (e.g., methanol, water, ethylene glycol, catalyst etc.) to encounter and react with the polymer chains. As illustrated in FIG. 2 (panel A), polymers including crystalline domains are less accessible to reactive solvent and catalyst compared to amorphous polymer. As further illustrated in FIG. 2 (panel B), however, polymers which are pre-treated via melt-processing can be substantially to completely amorphized, thus improving accessibility to reactive solvent and catalyst and improving the corresponding depolymerization process. Accordingly, samples which are not melt-processed, and thus retain regions of crystalline polymer, typically require higher temperatures (to disrupt the crystalline regions and allow access of solvent and catalyst) and/or longer depolymerization reaction times compared to melt-processed samples. Thus, melt-processing pre-treatment leads to a more efficient conversion of the condensation polymer to its corresponding monomers as desired depolymerization reaction products.

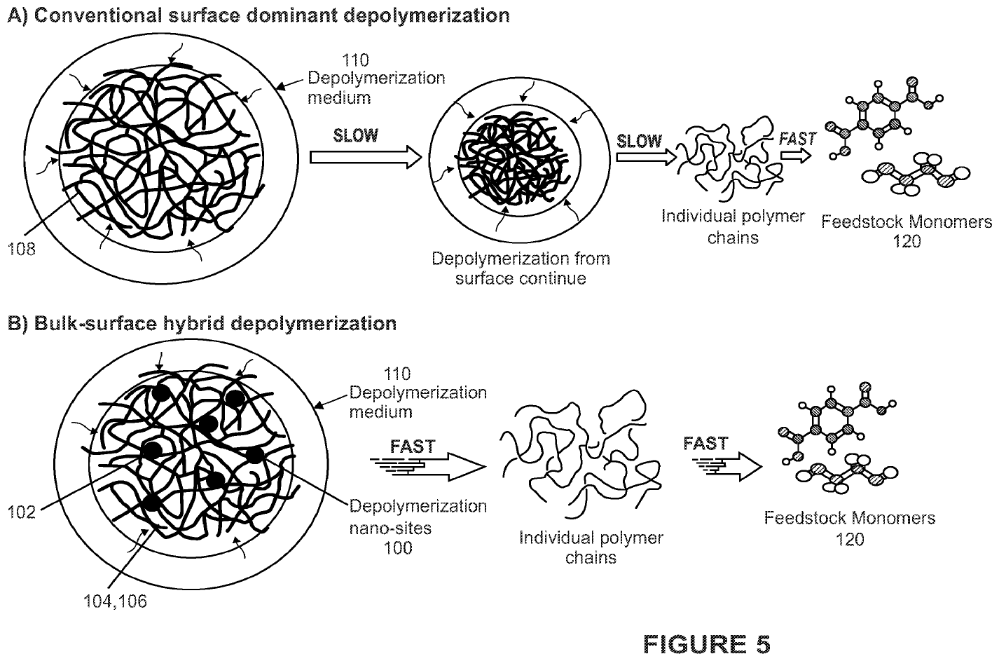

Furthermore, the inclusion of a catalyst at the melt-processing stage improves both the amorphization of the initial condensation polymer as well as the subsequent depolymerization of the polymer. During melt-processing, the catalyst can promote transesterification reactions (e.g., for a polyester condensation polymer), which can further reduce crystallinity beyond the reduction in crystallinity achieved by heating/melting alone, in particular when the melt-processed mixture further includes a monomer additive that is complementary to or reactive with the condensation polymer (e.g., 1,4-cyclohexane dimethanol (CHDM)) or other diol for a polyester condensation polymer). Without intending to be bound by theory, melt-processing PET or another condensation polymer with a catalyst, optionally with a suitable monomer additive that is complementary to or reactive with the condensation polymer, can generate reactive “nano-sites” 100 containing catalyst 102 and optionally monomer additives 104 embedded within the amorphized polymer 106 (FIG. 5, panel B), for example after melting and subsequent cooling of the condensation polymer feedstock. These reactive sites provide a route for depolymerization (e.g., forming corresponding depolymerization monomers 120) that, unlike conventional surface-dominant depolymerization as shown in FIG. 5, panel A, is not rate-limited by diffusion of catalyst and/or reactive solvent through the bulk polymer 108 from the depolymerization medium 110. The presence of these reactive sites can thus enable increased depolymerization rates.

In addition to its effect during melt-processing, the catalyst remains in the amorphized condensation polymer, which in turn enhances the rate and conversion of the depolymerization stage. For example, while the reactive solvent can be efficiently absorbed by and penetrate into solid phase amorphized condensation polymer in the reaction medium, transport of catalyst from the bulk reaction medium into the polymer is generally slow and can be rate-limiting for depolymerization. By incorporating the catalyst into the polymer during melt-processing (or “internal catalyst”), reactive solvent that penetrates into the solid polymer can react via catalysis from the internal catalyst without being rate-limited by the uptake of catalyst from the bulk reaction medium (or “external catalyst”).

Inclusion of the (internal) catalyst in the melt-processed mixture can improve amorphization even when the melt-processed mixture does not include a monomer additive that is complementary to or reactive with the condensation polymer (e.g., CHDM or otherwise). For example, the inclusion of a small amount of water during melt-processing can increase the amorphous content by partially hydrolyzing the starting condensation polymer, thereby reducing the overall molecular weight and increasing the relative number of polymer chain ends, which in turn increases the free volume within the polymer and reduces the overall crystallinity. For example, if the original condensation polymer feed consists only of PET, the presence of the catalyst along with some water can improve amorphization beyond the effect obtained by simply melting in the absence of the catalyst. Similarly, if the original condensation polymer feed consists of PET and PETG, then the presence of the catalyst can promote transesterification reactions that redistribute the CHDM units present in the original PETG to the original PET (e.g., forming a new polymer (e.g., “PTEG*”) that has a reduced amount of CHDM units compared to the original PETG). This randomization and redistribution effect reduces the overall crystallinity of the original blend (e.g., the original crystallinity of a PET/PETG blend). The melt-processed mixture can include 0.0001 wt. % to 1 wt. % water relative to the melt-processed mixture as a whole or the condensation polymer portion thereof.

The monomers corresponding to the amorphized condensation polymer generally include reaction products between the repeat units of the amorphized condensation polymer and the reactive solvent. For example, water can be selected as the reactive solvent for depolymerization via hydrolysis, forming diacid and diol monomers (for a polyester condensation polymer) or diacid and diamine monomers (for a polyamide condensation polymer). Similarly, a mono-alcohol (e.g., methanol) can be selected as the reactive solvent for depolymerization via alkylolysis (e.g., methanolysis), forming diester and diol monomers (for a polyester condensation polymer) or diester and diamine monomers (for a polyamide condensation polymer). In addition to the resulting monomers, the product mixture generally contains any reactive solvent not consumed during depolymerization as liquid medium containing the formed monomers therein, along with remaining catalyst (internal and/or external), other reaction medium components, etc.

### Condensation Polymers

Condensation polymers according to the disclosure can include polyesters, polyamides, or combinations thereof. The condensation polymers are generally thermoplastic polymers such as thermoplastic polyesters and polyamides. The melt-processed mixture can include a single condensation polymer along with a catalyst, or it can include two or more different condensation polymers along with the catalyst. For example, initial melt-processed mixture can include two or more polyesters, two or more polyamides, at least one polyester and at least one polyamide, etc. Examples of suitable polyamides include nylon 6,6, nylon 6, nylon 6,10, etc. Examples of other suitable thermoplastic polymers that can be melt-processed and amorphized according to the disclosure include polycarbonates, polyanhydrides, polyimides, polybenzimidazoles, polyquinoxlines, aromatic ladder polymers, phenol-formaldehyde polymers, urea-formaldehyde polymers, melamine-formaldehyde polymers, polyacetals, polyethersulfones, polyethers, polyphenylene oxides, polyarylenes, and thermoplastic polyurethanes.

The condensation polymer can include at least one polyester, such as polyethylene terephthalate (PET), polyethylene terephthalate glycol-modified (PETG; including typically about 5-50 mol. % or 15-30 mol. % cyclohexane dimethanol comonomer with 50-95 mol. % or 70-85 mol. % ethylene glycol comonomer), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polybutylene succinate terephthalate (PBST), polyethylene succinate (PES), poly(butylene succinate/terephthalate/isophthalate)-co-(lactate) (PBSTIL), liquid crystalline polyesters, or combinations thereof.

The condensation polymer can include, for instance, a blend of polyethylene terephthalate (PET) and polyethylene terephthalate glycol-modified (PETG). More generally, the condensation polymer can include a blend of condensation polymers, for example PET in combination with one or more copolyesters, such as PETG, isophthalate-modified copolyesters, sebacic acid-modified copolyesters, diethyleneglycol-modified copolyesters, triethyleneglycol modified-copolyesters, cyclohexanedimethanol modified-copolyesters, and/or polybutylene terephthalate. Such modified copolyesters generally have at least one of the TPA or EG units in PET at least partially replaced with modifying units (e.g., at least some terephthalic units replaced with isophthalic units, at least some ethylene glycol units replaced with diethyleneglycol units). Modified copolyesters may include, for example, 2-50 mol. %, 5-50 mol. %, 10-40 mol. %, 10-20 mol. %, 20-30 mol. %, or 15-30 mol. % of one or more modifying units. The copolyesters can have a low melting point or include an amorphous aromatic copolyester (such as one based on terephthalate/isophthalate copolymer with ethylene glycol or a copolyester made from a combination of terephthalic acid, ethylene glycol, and cyclohexyldimethanol). The condensation polymer can include 5-95 wt. % (e.g., at least 5, 10, 15, 20, 30, 40, 50, 60, 70, or 80 wt. % and/or up to 20, 30, 40, 50, 60, 70, 80, 90, or 95 wt. %) of PET, PETG, or other copolyesters relative to the total amount of condensation polymers in the initial melt-processed mixture. The disclosed method can be performed on combinations of polyesters or other condensation polymers in multilayer materials, for example laminated polyester structures (e.g., where different layers can have different polyester components or compositions).

The condensation polymer can further include 0.01-10 wt. % (e.g., at least 0.01, 0.1, 1, 2, 3, or 5 wt. % and/or up to 2, 3, 4, 6, 8, or 10 wt. %) of polylactic acid (PLA) (i.e., in addition to PET and PETG or other copolyester(s)) relative to the total amount of condensation polymers in the initial melt-processed mixture.

### Catalysts

Suitable catalysts for use in the method according to the disclosure include metal alkanoates, metal benzoates, metal carbonates, metal sulfates, acids, bases, and combinations thereof. Suitable metals in the various catalysts can include zinc, zirconium, titanium, sodium, potassium, etc. Example metal alkanoates include zinc acetate, zinc ethylhexanoate, and zinc fluoroacetate. Example metal benzoates include zinc benzoate, zinc chlorobenzoate, zinc bromobenzoate, zinc nitrobenzoate, and zinc methoxy benzoate. Example metal sulfates include sodium sulfate and potassium sulfate. Example metal carbonates include sodium carbonate and potassium carbonate. Example acid catalysts include sulfuric acid and sulfonic acid derivatives. Example bases include metal hydroxides such as sodium hydroxide and potassium hydroxide. The foregoing catalysts can be used as condensation catalysts for the condensation polymers described herein, for example also serving as a transesterification catalyst (e.g., when the condensation polymer includes a polyester) and/or a transamidation catalyst (e.g., when the condensation polymer includes a polyamide).

Suitable catalysts also include organic molecules which are metal-free or substantially metal-free, for example an organic base such as a nitrogen-containing amine, imine, amidine, or other cylclic tertiary amine base, for example including one or more (hetero)cyclic (alkyl) groups. Suitable amidine bases can be represented by R¹—C(=NR²)—NR³R⁴, where R¹-R⁴ can be independently selected to be alkyl groups (e.g., 1 to 10 carbon atoms) that are unsubstituted or substituted with one or more heteroatoms (e.g., N, O, S, P), R¹ and R³ can together form a cycloalkyl group (e.g., 3 to 12 carbon atoms) that are unsubstituted or substituted with one or more heteroatoms, and/or R² and R⁴ can together form a cycloalkyl group (e.g., 3 to 12 carbon atoms) that are unsubstituted or substituted with one or more heteroatoms. Particularly suitable catalysts include amidines such as 1,5,7-triazabicyclo[4.4.0]dec-5-ene (TBD) and 1,8-diazabicyclo[5.4.0]undec-7-ene (DBU). Other suitable nitrogen-containing organic catalysts include as 1,5-diazabicyclo[4.3.0]non-5-ene (DBN), 4-(dimethylamino)pyridine (DMAP), 1,4-diazabicyclo[2.2.2]octane (DABCO), N-methylimidazole (NMI), triethylenediamine, and dimethylamine (DMA). The foregoing catalysts can be used as condensation catalysts for the condensation polymers described herein, for example also serving as a transesterification catalyst (e.g., when the condensation polymer includes a polyester) and/or a transamidation catalyst (e.g., when the condensation polymer includes a polyamide). Furthermore, the foregoing catalysts can be substantially removed from the depolymerized reaction mixture via distillation, obviating the need for complex and energy-intensive filtration operations. For example, the organic base catalyst can have a boiling point in a range of 80-300° C. or 150-250° C., such as at least 80, 100, 120, 150, or 200° C. and/or up to 100, 120, 150, 170, 200, 250, or 300° C.(e.g., as a relatively volatile organic base catalyst). In embodiments, the method can further include recovering the organic base (or volatile) from the product mixture, and optionally performing a subsequent melt-processing of a new condensation polymer with the recovered organic base as the catalyst. Recovery of the organic base can be effected by distillation, heating, evaporation, or other separation processes.

The melt-processed mixture can include one or more catalysts in an amount in a range from 0.1 wt. % to 20 wt. % or 1 wt. % to 5 wt. % relative to the melt-processed mixture, including the total amount of (amorphized) condensation polymer(s) and catalyst. More generally, the catalyst can be present in amount of at least 0.1, 0.2, 0.5, 1, or 2 wt. % and/or up to 2, 4, 5, 6, 8, 10, 12, 15, or 20 wt. % relative to the melt-processed mixture. Alternatively or additionally, the amount of catalyst can be expressed relative to the total amount of (amorphized) condensation polymer(s) only (e.g., excluding catalyst weight and any other additive such as comonomers). The catalyst added to the melt-processed mixture generally remains in the mixture after melt-processing, so it is predominantly present in the amorphous feed material in corresponding amounts relative to the amorphous feed material.

The catalyst can be heat-stable at temperatures experienced during the melt-processing. Suitably, the catalyst added to the mixture for melt-processing is heat-stable or heat-resistant so that it does not degrade or otherwise lose its activity during melt-processing. This permits the catalyst to remain in the amorphous feed material as an active internal catalyst that also can catalyze depolymerization in the next step. For example, after exposure to the high melting temperatures during melt processing, , the catalyst suitably is present in an amount of at least 20, 50, 85, 90, 95, or 98 wt. % and/or up to 90, 95, 99, or 100 wt. % relative to its initial amount (indicating that substantial amounts of the catalyst do not decompose or degrade to other components upon heating). Alternatively or additionally, after exposure to the high melting temperatures during melt processing, the catalyst suitably retains at least 20, 50, 80, 85, 90, 95, or 98% and/or up to 90, 95, 99, or 100% of its initial catalytic activity (indicating that substantial amounts of the catalyst are not inactivated upon heating). As a class of catalysts, metal benzoates (e.g., substituted or unsubstituted benzoates such as zinc benzoates) are particularly heat-stable and useful at higher melt-processing temperatures according to the disclosure (e.g., 280° C. and higher), for example relative to zinc acetate.

### Monomer Additives

The melt-processed mixture can further include a monomer that can be reactive with the condensation polymer. Such an additional monomers can be compounded or mixed with the initial condensation polymer during melt-processing (e.g., in an extruder). The added monomers generally include compounds that are complementary to or reactive with repeat units of the condensation polymer. For example, the added monomers can include diols (or polyols) or diacids (or polyacids) for addition to polyester condensation polymers (i.e., reactive with the diacid or diol units thereof, respectively), or diamines (or polyamines) or diacids (or polyacids) for addition to polyamide condensation polymers (i.e., reactive with the diacid or diamine units thereof, respectively). The added monomers can be incorporated into the condensation polymers via transesterification or transamidation, etc., which are catalyzed by the catalyst in the mixture at the high melt-processing temperature. The added monomers can be selected to reduce the crystallinity of the amorphized condensation polymer in which they are incorporated, or to impart additional functionality to the amorphized condensation polymer. Examples of added monomer include diethylene glycol (DEG), 1,4-cyclohexane dimethanol (CHDM), bis(hydroxymethyl)tricyclo[5.2.1.0^{2,6}]decane (BHTD), and bis(2-hydroxyethyl) terephthalate (BHET). For example, BHET can be added to PET to provide more hydroxyl groups and reduce PET molecular weight (e.g., via chain scission during transesterification).

Suitable diols can be represented by HO—R—OH (where R includes alkyl, cyclic, and/or aryl groups). The R group can have 2-40 carbon atoms (e.g., at least 2, 4, 6, 8 or 10 and/or up to 4, 8, 12, 20, 30, or 40 carbon atoms) and can further include one or more heteroatoms such as O, N, S, etc. Suitable polyols include glycerol, ethylene glycol, diethylene glycol, triethylene glycol, tetraethylene glycol, propylene glycol, dipropylene glycol, tripropylene glycol, 1,3-propanediol, 1,3-butanediol, 1,4-butanediol, neopentyl glycol, 1,6-hexanediol, 1,8-octanediol, 1,4-cyclohexanedimethanol, glycerol, trimethylolpropane, 1,2,6-hexanetriol, pentaerythritol, (meth)acrylic polyols, isosorbide, and combinations thereof. Higher-functional polyols such as triols etc. can be useful to reduce crystallinity.

Suitable diacids can be represented by HOOC—R—COOH (where R includes alkyl, cyclic, and/or aryl groups). The R group can have 2-40 carbon atoms (e.g., at least 2, 4, 6, 8 or 10 and/or up to 4, 8, 12, 20, 30, or 40 carbon atoms) and can further include one or more heteroatoms such as O, N, S, etc. Specific examples of dicarboxylic acids or polyacids include adipic acid, sebacic acid, isophthalic acid, phthalic acid, 2,6-naphthalenedicarboxylic acid, 4,4′-biphenyldicarboxylic acid, 4,4′-oxybisbenzoic acid, 5-sulfoisophthalic acid monosodium salt, 1,4-butanedioic acid, 1,6-hexanedioic acid, decanedioic acid, 1,4-cyclohexanedicarboxylic acid.

Suitable diamines can be represented by H₂N—R—NH₂ (where R includes alkyl, cyclic, and/or aryl groups). The R group can have 2-40 carbon atoms (e.g., at least 2, 4, 6, 8 or 10 and/or up to 4, 8, 12, 20, 30, or 40 carbon atoms) and can further include one or more heteroatoms such as O, N, S, etc. Specific examples of diamines or polyamines include 1,6-hexanediamine, ethylene diamine, toluene diamine, diaminodiphenylmethane and polymethylene polyphenylene polyamines, aminoalcohols, etc. The amino functional group can be primary, secondary, or tertiary. A specific example of a suitable polyetheramine is polyoxypropylenediamine (e.g., JEFFAMINE D-400 as a diamine). Higher-functional polyamines such as triamines etc. as well as a branched polyamines can be useful to reduce crystallinity.

Added monomers can be present in the melt-processed mixture in an amount in a range from 0.5 wt. % to 90 wt. %, 5 wt. % to 30 wt. %, or 0.5 wt. % to 5 wt. % relative to the initial melt-processed mixture. For example, the monomer can be present in the melt-processed mixture in amount of at least 0.5, 1, 2, 5, 8, 10, 15, 20, or 30 wt. % and/or up to 5, 10, 15, 20, 25, 30, 50, 70, or 90 wt. % relative to the initial melt-processed mixture. The monomer added to the melt-processed mixture is generally incorporated into the amorphous condensation polymer as a repeat unit therein via catalyzed reaction with the initial condensation polymer, although some monomers can remain unreacted, and/or some monomers can remain as end units on a polymer chain. The combined amount of condensation polymer(s), catalyst, and monomers (when present) can correspond to at least 95, 98, 99, or 99.5 wt. % and/or up to 97, 98, 99, 99.5, 99.8, or 100 wt. % of the (initial) melt-processed mixture.

### Crystalline/Amorphous Polymer Content

The condensation polymer in the melt-processing mixture (e.g., the condensation polymer as initially present therein or added thereto) typically has a high crystalline polymer content and/or a low amorphous polymer, for example being representative of a polymer feedstock to be recycled, but which would otherwise not be particularly amenable to chemical recycling/depolymerization (i.e., as discussed above) without melt-processing as described herein. For example, the condensation polymer in the melt-processing mixture (e.g., the condensation polymer as initially present therein or added thereto) can have a crystalline polymer content of 20 wt. % or more. Suitably, the amorphized condensation polymer in the amorphous feed material (e.g., after melt-processing and/or quenching, but before depolymerization) can have a crystalline polymer content of 15 wt. % or less. More generally, the condensation polymer in the initial melt-processing mixture can have a crystalline polymer content of at least 5, 10, 20, 25, 30, 35, 40, 45, 50, 60, 70, or 80 wt. % and/or up to 10, 20, 30, 40, 50, 60, 70, 80, 90, or 100 wt. %. Alternatively or additionally, the condensation polymer in the initial melt-processing mixture can have an amorphous polymer content of at least 1, 2, 5, 10, 20, 30, 40, 50, 60, 70, 80, or 90 wt. % and/or up to 20, 30, 40, 50, 60, 70, 80, 90, 95, or 98 wt. %. Alternatively or additionally, the amorphous condensation polymer in the amorphous feed material can have a crystalline polymer content of at least 0.1, 0.2, 0.5, 1, 2, 4, 6, 8, 10, 12, 15, or 20 wt. % and/or up to 1, 2, 3, 5, 7, 10, 15, 20, 25, or 30 wt. %. Alternatively or additionally, the amorphous condensation polymer in the amorphous feed material can have an amorphous polymer content of at least 60, 65, 70, 75, 80, 85, 90, 95, 98, or 99 wt. % and/or up to 70, 75, 80, 90, 95, 98, 99, 100 wt. %. Alternatively or additionally, a difference between the crystalline polymer content of the condensation polymer in the initial melt-processing mixture and the amorphous feed material can be at least 1, 2, 5, 10, 15, 20, or 30 wt. % and/or up to 5, 10, 20, 30, 40, 50, 60, 70, or 80 wt. % (i.e., reflecting a reduction in crystalline content upon melt-processing). Alternatively or additionally, a difference between the amorphous polymer content of the condensation polymer in the amorphous feed material and the initial melt-processing mixture can be at least 1, 2, 5, 10, 15, 20, or 30 wt. % and/or up to 5, 10, 20, 30, 40, 50, 60, 70, or 80 wt. % (i.e., reflecting an increase in amorphous content upon melt-processing). The foregoing ranges for crystalline and amorphous polymer content can apply to single condensation polymers in the mixtures being processed and/or collectively to all condensation polymers in the mixtures being processed (e.g., average crystalline and/or amorphous polymer contents for the processed blends).

### Melt-Processing

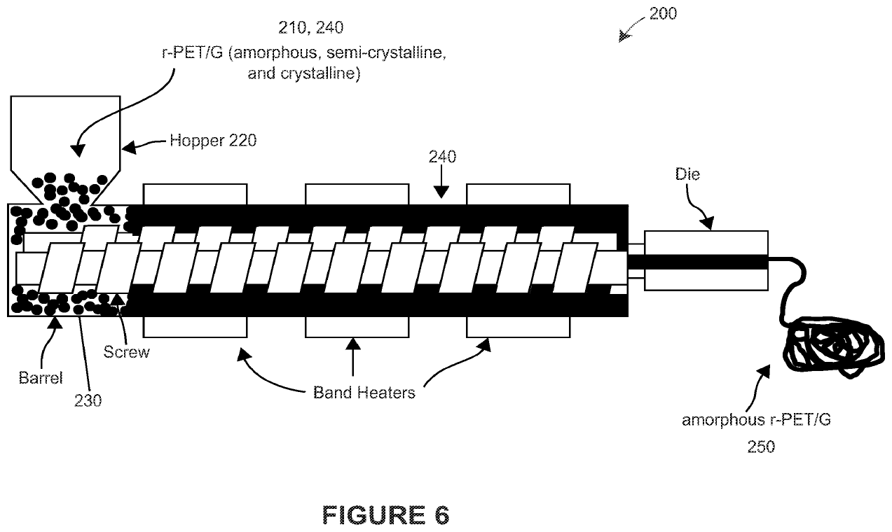

In general, melt-processing according to the disclosure comprises heating a mixture of the condensation polymer, the catalyst and optionally other additives to a temperature sufficiently high to the condensation polymer (e.g., but low enough to avoid thermal degradation). Any apparatus typically used for melt-processing of condensation polymers may be used, for example twin- and single-screw extrusion apparatuses operated at a sufficiently high temperature to melt the base condensation polymer material. A typical extrusion apparatus 200 is illustrated in FIG. 6. Unprocessed condensation polymer feed 210 comprising a mix of amorphous, semi-crystalline, and crystalline polymer is delivered via a hopper 220 to a twin- or single-screw extruder 230 that is maintained at a temperature sufficient to melt the polymer. The catalyst 240 as well as any monomer additives (not shown) can be added to the extruder 230 along with the condensation polymer feed 210 and/or at one or more downstream points along the extruder (e.g., via an injection port along the extruder 230 barrel, not shown). The screw ensures mixing of the polymer melt while pushing melt-processed polymer 250 through the barrel of the extruder towards the exit. The melt-processed polymer 250 exits the extruder through a die and is converted to a filament which is subsequently chopped and milled to form a powder of amorphous polymer including the catalyst therein. Melt-processing temperatures in a range of 100° C. to 400° C., 200° C. to 300° C., 260° C. to 320° C., or 280° C. to 300° C., for example at least 100, 150, 170, 200, 220, 250, 280, or 300° C. and/or up to 160, 180, 210, 240, 280, 300, 320, 350, or 400° C., are particularly suitable for polyester condensation polymers such as PET and/or PETG. Alternatively or additionally, the melt-processing temperature can be expressed relative to the highest melting temperature (Tm) for the condensation polymer(s) in the melt-processed mixture. For example, if the melt-processed mixture includes two polymers, one with a melting point of 260° C. and another with a melting point of 280° C., then the highest melting temperature (Tm) is 280° C. Suitably, The melt-processing temperature can be at least 5, 10, 15, 20, 25, 40, 60, or 100° C. above the highest melting temperature (Tm) and/or up to 10, 15, 25, 35, 50, 65, 80 or 100° C. above the highest melting temperature (Tm).

The method according to the disclosure further can include quenching the melt-processed amorphous feed material in a liquid medium prior to depolymerizing. The quenching in a (relatively) cold liquid medium after extrusion or other melt-processing can be used to form filaments or strands of the amorphous feed material to limit or prevent crystal growth therein prior to depolymerization. The liquid medium for quenching can be the same material(s) used for the reaction medium. A suitable quenching temperature is below the glass transition temperature (Tg) of the melt-processed polymer or polymer blend, for example at least 5, 10, 15, or 20° C. below the Tg of the melt-processed polymer and/or in a range of 0-60° C., 10-50° C., or 20-40° C. Alternatively or additionally, the quenching temperature can be below the crystallization temperature (Tc) of the melt-processed polymer or polymer blend, for example at least 5, 10, 15, or 20° C. below Tc.

### Reactive Solvents

In embodiments, the reactive solvent can include one or more of water, mono-alcohols (e.g., methanol, ethanol), diols, mono-amines, diamines, and combinations thereof. In addition to mono-alcohols, diols or polyols such as ethylene glycol, glycerols, and those mentioned above as monomer additives can be used. Suitable amino compounds can include mono-amines, diamines and polyamines (e.g., as mentioned above as monomer additives), ammonia, and alcoholic amines (e.g., an amino alkanol). The reaction medium and/or the reactive solvent portion thereof can be present in a relative amount of at least 2, 5, 7, 10, 12, 15, or 20 and/or up to 6, 8, 10, 15, 20, 30, 40, or 50, on a w/w or v/v basis, relative to the amount of amorphous feed material. The reaction medium can optionally include non-reactive solvents such as organic non-alcohol or non-amine solvents (e.g., non-protic or aprotic solvents such as tetrahydrofuran, chloroform, etc.) to speed up the depolymerization by enhancing the swelling the of the amorphous polymer (e.g., PET/G). In addition to increasing the rate of depolymerization (e.g., via polymer swelling), the addition of non-reactive solvents can be used to enable phase-separation and recovery of the reaction products (e.g., using fluorinated organic solvents, ionic liquids, etc.). When included, the non-reactive solvent can similarly be included in a relative amount of at least 2, 5, 7, 10, 12, 15, or 20 and/or up to 6, 8, 10, 15, 20, 30, 40, or 50,on a w/w or v/v basis, relative to the amount of amorphous feed material.

In some embodiments when the condensation polymer (or likewise, the amorphized condensation polymer) includes a polyester, the reactive solvent can include water (e.g., for depolymerization via hydrolysis), and the monomers in the product mixture can include diacids and diols formed from the repeat units of the polyester. For example, if the original condensation polymer is PET, then the corresponding monomers formed by depolymerization via hydrolysis include terephthalic acid (TPA) and ethylene glycol (EG). If a monomer is added to the melt-processed mixture, then such monomer can also be recovered in the product mixture. For example, if the original condensation polymer is PET which is melt-processed with CHDM and then depolymerized via hydrolysis, then the corresponding monomers include TPA, EG, and CHDM.

In some embodiments when the condensation polymer (or likewise, the amorphized condensation polymer) includes a polyester, the reactive solvent can include methanol (e.g., for depolymerization via methanolysis; or mono-alcohol more generally), and the monomers in the product mixture can include diesters and diols formed from the repeat units of the polyester. For example, if the original condensation polymer is PET, then the corresponding monomers formed by depolymerization via methanolysis include dimethyl terephthalate (DMT) and ethylene glycol (EG). If a monomer is added to the melt-processed mixture, then such monomer can also be recovered in the product mixture. For example, if the original condensation polymer is PET which is melt-processed with CHDM and then depolymerized via methanolysis, then the corresponding monomers include DMT, EG, and CHDM. Analogous ethyl or other alkyl esters can be formed when using mono-alcohols other than methanol, such as ethanol, etc.

In some embodiments when the condensation polymer (or likewise, the amorphized condensation polymer) includes a polyester, the reactive solvent can include ethylene glycol (e.g., for depolymerization via glycolysis; or diol more generally) and the monomers in the product mixture can include diesters formed from the repeat units of the polyester. For example, if the original condensation polymer is PET, then the corresponding monomers formed by depolymerization via glycolysis include bis(hydroxymethyl)terephthalate (BHET). If a monomer is added to the melt-processed mixture, then such monomer can also be recovered in the product mixture. For example, if the original condensation polymer is PET melt-processed with CHDM and then depolymerized via glycolysis, then the corresponding monomers include BHET and CHDM.

In some embodiments when the condensation polymer (or likewise, the amorphized condensation polymer) includes a polyamide, the reactive solvent can include water (e.g., for depolymerization via hydrolysis), and the monomers in the product mixture can include diacids and diamines formed from the repeat units of the polyamide.

In some embodiments when the condensation polymer (or likewise, the amorphized condensation polymer) includes a polyamide, the reactive solvent can include methanol, (e.g., for depolymerization via methanolysis), and the monomers in the product mixture can include diesters and diamines formed from the repeat units of the polyamide.

In some embodiments when the condensation polymer (or likewise, the amorphized condensation polymer) includes a polyamide, the reactive solvent can include ethylene glycol (e.g., for depolymerization via glycolysis), and the monomers in the product mixture include diesters and diamines formed from the repeat units of the polyamide.

Suitably, the initial melt-processed mixture is free or substantially free of reactive solvents (i.e., before such reactive solvents are added to the amorphous feed material for depolymerization). For example, the initial melt-processed mixture typically contains not more than 2, 1, 0.5, 0.2, or 0.1 wt. % of such reactive solvents (e.g. water, methanol, or other mono alcohols). However, the melt-processed mixture can include, for example, 0.0001 wt. % to 1 wt. % water to promote transesterification. Small amounts of water can be due to naturally present or ambient moisture (e.g., no added water) or, alternatively, some amount of added water. For example, the initial melt-processed mixture can include at least 0.0001, 0.001, 0.01, 0.1, or 0.2 wt. % and/or up to 0.1, 0.2, 0.4, 0.6, 0.8, or 1 wt. % water relative to the melt-processed mixture as a whole or the condensation polymer portion thereof. When some water is present in the melt-processed mixture, the mixture is suitably free or substantially free of other reactive solvents in general and/or free or substantially free of added water (i.e., non-ambient or non-environmental water).

### External Catalysts

In various embodiments, the reaction medium can either further include an external catalyst or be substantially free from external catalysts. For example, in some cases, it is possible to include sufficient internal catalyst at the time of melt-processing so that depolymerization can proceed simply by heating the amorphized condensation polymer in a reaction medium with reactive solvent but without further catalyst. In general, however, it can be desirable to include an external catalyst in the reaction medium to assist or accelerate depolymerization by directly acting on the exposed external surfaces of the amorphized condensation polymer. The external catalyst can generally be selected from the same options as described for the (internal) catalyst added to the melt-processed mixture and incorporated into the amorphous feed material. The internal catalyst and the external catalyst can be the same catalysts or different catalysts. When an external catalyst is present, the external catalyst can be present in the reaction medium in an amount in a range from 0.1 wt. % to 10 wt. % or 0.5 wt. % to 5 wt. % relative to the reaction medium, for example in an amount of at least 0.1, 0.2, 0.5, 1, or 2 wt. % and/or up to 1.5, 3, 5, 7, or 10 wt. %. If no external catalyst is added to the reaction medium, the reaction medium suitably does not contain more than 0.001, 0.01, or 0.1 wt. % of an external catalyst (e.g., the same catalyst species as included for the internal catalyst and/or all catalyst species combined).

### Surfactants

The reaction medium can further include a surfactant. A surfactant can lower the surface energy of the depolymerization reaction medium to improve polymer wetting and depolymerization efficiency. The surfactant suitably can be included in an amount of 0.1-10 wt. % or 0.5-4 wt. % in the reaction medium. The surfactant is not particularly limited, for example including anionic, cationic, zwitterionic, and non-ionic surfactants. Examples of suitable anionic surfactants include sodium dodecyl sulfate (SDS) and sodium lauryl sulfate (SLS).

### Swelling Treatment

The method can further include swelling the amorphized condensation polymer with at least one of a gaseous swelling and a non-protic solvent swelling agent (e.g., to enhance depolymerization). High-pressure gases such as carbon dioxide and oxygen can swell condensation polymers (e.g., PET, PETG, and other polyesters), making the amorphous regions of the polymer more accessible during depolymerization. For example, after melt-processing and prior to depolymerization, the amorphized condensation polymer can be exposed to carbon dioxide and/or oxygen at high pressures and above the Tg of the polymer, for example in a pressurized vessel (e.g., which could be the reaction vessel for depolymerization, but without added reaction solvent/reaction medium). The pressure is then released, the swelled amorphized condensation polymer is then added to or otherwise combined with the reaction solvent/reaction medium, and depolymerization can then be performed. As described above, non-protic or aprotic solvents such as tetrahydrofuran, chloroform, etc. can be added to the reaction medium as an additional or alternative means to swell the amorphized condensation polymer to enhance depolymerization.

### Auxiliary Catalysts

The reaction medium can further include at least one of polystyrene (PS) sulfonic acid beads and a hydroxylated pyridine. The PS sulfonic acid beads can be included as a solid acid external catalyst that can be easily recovered and re-used after deopolymerization. The hydroxylated pyridine can serve as a bifunctional catalyst.

### Depolymerization

Depolymerization can include heating the amorphous feed material in the reaction medium to a temperature in a range of, for example, 120° C. to 200° C. Any reaction vessel typically used for batch, semi-batch, or continuous reactions may be used. Temperatures from at least 25° C., 60° C., 120° C., or 150° C.and/or up to 50° C., 75° C., 100° C., 150° C., 170° C., or 200° C. are particularly suitable for depolymerizing polyester condensation polymers such as PET and/or PETG. While depolymerization is often performed at elevated temperatures (e.g., above 100° C.), including catalysts such as sodium amide, sodium hydroxide, etc. can enable depolymerization to proceed at lower temperatures, such as temperatures as low as ambient (room) temperature. The depolymerization reaction is suitably performed for a sufficient time (or residence time in a continuous reactor) to essentially completely depolymerize the (amorphized) condensation polymer into its corresponding monomers, for example, at least 0.01, 0.1, 0.5, 1, 2, 3, or 4 hr and/or up to 2, 4, 6, 8, 10, 12, 18, or 24 hr. Suitably, the product mixture contains 20, 10, 5, 2, 1, 0.5, 0.2, or 0.1 wt. % or less of the (amorphized) condensation polymer relative to the initial condensation polymer, for example corresponding to a conversion of least 80, 90, 95, 98, or 99 wt. % and/or up to 95, 98, 99, or 100 wt. % of the condensation polymer.

In a refinement, the condensation polymer includes at least one of polyethylene terephthalate (PET) and polyethylene terephthalate glycol-modified (PETG); the reactive solvent comprises methanol; the monomers in the product mixture comprise dimethyl terephthalate (DMT), ethylene glycol (EG), and optionally 1,4-cyclohexane dimethanol (CHDM) (e.g., when PETG is an included condensation polymer and/or when CHDM is an added monomer to the melt-processed mixture); and the method further comprises hydrogenating the DMT to form (further) CHDM. Suitable hydrogenation processes are generally known in the art, for example by reaction with hydrogen gas and a suitable catalyst. For example, hydrogenation of DMT can be performed in two steps to first convert DMT to 1,4-cyclohexane dimethylcarboxylate (CHDC), and then convert CHDC to CHDM.

### Organic Catalysts

A challenge associated with chemical recycling of condensation polymers by depolymerization is efficient removal of catalyst from the recycled monomer stream. Metal-based catalysts such as zinc salts generally must be removed by filtration. Accordingly, further improvements to depolymerization processes can be achieved by using a catalyst which can be easily removed, such as an organic catalyst, in particular an organic base catalyst as described above, which can be removed by distillation and then recycled/re-used as an internal catalyst in subsequent melt-processing of new condensation polymers to be chemically recycled.

### EXAMPLES

The following examples illustrate the disclosed methods, but are not intended to limit the scope of any claims thereto.

Zinc acetate (ZnAc₂), zinc 2-ethylhexanoate (ZnEH), bis(2-hydroxyethyl)terephthalate (BHET), 1,4-cyclohexanedimethanol (CHDM), 4,8-bis(hydroxymethyl)tricyclo[5.2.1.0^{2,6}]decane (BHTD), 1,5,7-triazabicyclo[4.4.0]dec-5-ene (TBD), 1,8-diazabicyclo[5.4.0]undec-7-ene (DBU), dimethyl terephthalate (DMT), ethylene glycol (EG), and methanol were acquired from commercial sources and used as received. Post-consumer PET bottles (SP code #1) were shredded, cleaned with acetone or a surfactant solution, dried, and ground into a powder prior to use.

### Melt-Processing

PET was subjected to melt-processing pre-treatment by heating in an extruder. For samples containing an internal catalyst, ground PET was mixed with a solution containing the catalyst and dried. Extrusion of ground PET was performed using a DSM Xplore 15cc Micro Extruder equipped with a co-rotating conical twin screw; any suitable extruder can also be used. PET and optionally a diol monomer additive were compounded at 280° C. for 4 min; the melt-processed PET was extruded through a dye into a cooled methanol bath to minimize crystal re-growth upon cooling. The resulting extruded PET filaments were isolated and dried under vacuum overnight.

Depolymerization reactions were run by charging a reaction mixture including PET, catalyst, and reactive solvent to a high-pressure reaction flask and holding the mixture at a fixed temperature. Prior to depolymerization, the PET can optionally be pre-treated by melt-processing as described above. To achieve 100% depolymerization, the depolymerization reaction was run until the reaction mixture was visibly clear. In some experiments, depolymerization was quenched prior to reaching 100% depolymerization by rapidly cooling the reaction mixture by addition of water after a fixed reaction time. For these experiments, an amount of non-depolymerized PET was determined by filtering the quenched reaction mixture, rinsing the water-insoluble residue (i.e. non-depolymerized PET), briefly rinsing the residue with chloroform, and drying and weighing the residue. A depolymerization yield was then calculated according to:

Depolymerization Yield (%)=100%−(Residue Weight)/(Initial Polymer Weight)

Reaction mixtures resulting from depolymerization reactions were dissolved in chloroform and filtered, and the filtrates were characterized by ¹H NMR, ¹³C NMR, and GC-MS to confirm the presence of monomers of corresponding to repeat units of the parent condensation polymers. For example, reaction mixtures resulting from depolymerization of PET by methanolysis were confirmed by NMR and GC-MS to include dimethylterephthalate and ethylene glycol.

NMR spectra were acquired using a 500/54 Premium Shielded Spectrometer (Agilent Technologies). LC-MS measurements were performed using a chromatograph equipped with a 2.1 mm×100 mm Waters Acquity BEH-C18 UPLC column. DSC data were acquired using a TA Q100 differential scanning calorimeter; DSC scans were performed over the range of −80° C. to 300° C.at a scan rate of 10° C./min.

### Example 1: Solvent:Polymer Ratio

Initial depolymerization experiments were performed to determine an optimal amount of reactive solvent to use in the depolymerization process, such as an optimal amount of methanol to use for depolymerization by methanolysis. Table 1 shows the depolymerization yield of samples of r-PET/G using various amounts of methanol as the reactive solvent. Specifically, 0.3 g of r-PET/G which had been melt-processed with 2.5 wt. % of zinc acetate (internal catalyst) was reacted at 160° C.for 3 hr in varying amounts of methanol. As seen in Table 1, 100% depolymerization within 3 hr was obtained for reaction systems containing 2.0 mL or 3.0 mL of methanol per 0.3 g of starting polymer. Using less than 2.0 mL of solvent resulted in less than 100% depolymerization within 3 hr, possibly due to reduced mobility of reactants within the reaction mixture. Using greater than 3.0 mL solvent also resulted in less than 100% depolymerization within 3 hr, possibly due to dilution effects.

TABLE 1

| | Methanol (mL) per 0.3 g of r-PET/G | | | | | | |
|---|---|---|---|---|---|---|---|
| | 0.5 | 1.0 | 2.0 | 3.0 | 4.0 | 5.0 | 6.0 |
| Depolymerization | 56% | 93% | 100% | 100% | 95% | 90% | 85% |
| Yield (3 hr @ | | | | | | | |
| 160° C.) | | | | | | | |

### Example 2: Depolymerization Temperature

Table 2 shows the time required to fully depolymerize samples of melt-processed PET at various reaction temperatures. For each reaction temperature listed, 0.3 g of PET was melt-processed with 2.5 wt. % zinc acetate (internal catalyst) at 280° C. for 4 min, and the resulting polymer was reacted in 2.5 mL methanol with an additional 2.0% zinc acetate (external catalyst) at the stated reaction temperature until the mixture was visibly clear, indicating complete depolymerization. As indicated in Table 2, depolymerization proceeded more quickly at higher temperature; when the reaction was carried out at 100 ° C., depolymerization was incomplete after 5 days. Depolymerization at 160° C. was completed in 3 hr; this reaction time provided a good basis for comparing the effects of other reaction parameters (e.g., catalyst type and loading, reactive monomer type and loading),

TABLE 2

| | Reaction Temperature | | | | | |
|---|---|---|---|---|---|---|
| | 200° C. | 180° C. | 160° C. | 140° C. | 120° C. | 100° C. |
| Time to Fully | 1 hr | 1.5 hr | 3 hr | 9 hr | 26 hr | >120 hr |
| Depolymerize | | | | | | |

### Example 3: Melt-Processing

Table 3 lists depolymerization yields for samples of non-melt-processed and melt-processed PET at various reaction temperatures. Samples of PET which had not been melt-processed (top row) and samples of PET which had been melt-processed at 280° C. for 4 min without an internal catalyst (bottom row) were depolymerized via methanolysis in the presence of an external catalyst. Specifically, 0.3 g of non-melt-processed or melt-processed PET, 3.0 mL of methanol, and 2.0 wt. % zinc acetate external catalyst were reacted for 3 hr at fixed temperatures ranging from 140° C. to 200° C. As seen in Table 3, at each reaction temperature, the melt-processed PET sample was more readily depolymerized than the non-melt-processed PET sample. The higher depolymerization yields of the pre-treated PET samples suggest that melt-processing of PET amorphized the polymer and enabled easier access of catalyst and reactive solvent during depolymerization compared to non-pre-treated PET.

TABLE 3

| | Depolymerization Yield (3 hr) | | | |
|---|---|---|---|---|
| | 140° C. | 160° C. | 180° C. | 200° C. |
| Non-Melt-Processed PET | 9% | 34% | 37% | 42% |
| Melt-Processed PET | 41% | 72% | 86% | 95% |

### Example 4: Melt-Processing with Internal Catalyst

PET samples that had been melt-processed in the presence of a catalyst (i.e., an internal catalyst) were also subjected to depolymerization via methanolysis. Table 4 shows the depolymerization yields of samples of PET which had been melt-processed with 2.5 wt. % zinc acetate (i.e., internal catalyst). PET samples were depolymerized by methanolysis in 2.0 mL methanol and 2.0 wt. % zinc acetate external catalyst at 160 ° C. for fixed reaction times, as indicated. Table 4 shows the progression of depolymerization over time; depolymerization was essentially complete within 3 hr. It can also be seen from comparing Tables 3 and 4 that including an internal catalyst during melt-processing improved depolymerization efficiency compared to when an internal catalyst was not included. Table 4 indicates that depolymerization of a PET sample which was melt-processed in the presence of zinc acetate as an internal catalyst was completely depolymerized after 3 hr of methanolysis at 160° C., while Table 3 indicates that a PET sample which was melt-processed without an internal catalyst was only 72% depolymerized after 3 hr of methanolysis at 160° C., and a PET sample which was depolymerized without melt-processing was only 34% depolymerized after 3 hr of methanolysis at 160° C. Thus, while melt-processing alone improved depolymerization, the further inclusion of an internal catalyst during melt-processing substantially improved depolymerization even further.

TABLE 4

| | Reaction Time at 160° C. | | | | | |
|---|---|---|---|---|---|---|
| | 0.5 hr | 1.0 hr | 1.5 hr | 2.0 hr | 2.5 hr | 3.0 hr |
| Depolymerization | 14% | 49% | 69% | 90% | 95% | 100% |
| Yield | | | | | | |

### Example 5: External Catalyst

Increasing the level of catalyst present during depolymerization (i.e., external catalyst) also improves depolymerization efficiency. Table 5 shows the time required to fully depolymerize samples of PET that had been melt-processed with 2.5 wt. % zinc acetate as internal catalyst in the presence of varying levels of additional zinc acetate (i.e., external catalyst). Specifically, 0.3 g of melt-processed PET was reacted in 2.0 mL of methanol at 160° C. in the presence of the stated levels (wt. % based on PET) of additional zinc acetate as an external catalyst. Increasing the external catalyst level from 1 wt. % to 2 wt. % significantly accelerated the depolymerization; the effect of further increasing the external catalyst above 2 wt. % was less pronounced.

TABLE 5

| | Zinc acetate (wt. %) as external catalyst | | | | | |
|---|---|---|---|---|---|---|
| | 1.0 | 2.0 | 3.0 | 4.0 | 5.0 | 6.0 |
| Time to Fully Depolymerize | 480 | 180 | 175 | 170 | 135 | 120 |
| at 160° C. (min) | | | | | | |

### Example 6: Thermal Properties

Thermal properties of non-melt-processed and melt-processed PET samples were evaluated using DSC. Crystallization temperatures (Tc) and glass transition temperatures (Tg) of PET samples that were not melt-processed, melt-processed without internal catalyst, or melt-processed with either zinc acetate (ZnAc₂) or zinc ethylhexanoate (ZnEH) as an internal catalyst were determined by DSC; results are shown in Table 6. Melt-processing of PET without an internal catalyst resulted in polymer with a ˜20° C. lower Tm compared to PET which was not-melt-processed, suggesting that melt-processing alone led to a reduction in molecular weight. Incorporating zinc acetate or zinc ethylhexanoate as an internal catalyst led to a further ˜10° C. and ˜13° C. reduction, respectively, in both Tc and Tg compared to PET that was melt-processed without an internal catalyst, suggesting that each internal catalyst provided further molecular weight reduction during melt-processing.

TABLE 6

| Internal Catalyst | Tc | Tg |
|---|---|---|
| N/A (not melt-processed) | 140° C. | 81° C. |
| Melt-processed without internal catalyst | 119° C. | 78° C. |
| Melt-processed with 2.5% ZnAc₂ | 109° C. | 68° C. |
| Melt-processed with 4.7% ZnEH | 106° C. | 65° C. |

### Example 7: Internal Catalyst and Monomer Additive Effect on Thermal Properties

Examples 7-9 examine the effects of different internal catalysts as well as the optional inclusion of a monomer additive during initial melt-processing of PET. Table 7 lists the internal catalyst, monomer additive, and thermal properties of samples S0-S13. In Table 7, the amounts of internal catalyst monomer additive are expressed as mol. % relative to moles of repeat units in the PET. Thermal properties of the series of PET samples listed in Table 7 were measured by DSC to evaluate the effects of including internal catalyst and/or a monomer additive during melt-processing. S0 was non-melt-processed PET, and S1 was PET that was melt-processed without any additives (i.e., no internal catalyst or monomer additive). S2-S13 were prepared by dissolving the internal catalyst and/or monomer additive listed in Table 7 in a solvent, mixing the solution with PET granules, evaporating the solvent, and melt-processing the resulting granules at 280° C. for 4 minutes followed by immediate quenching in cold methanol to prevent crystal re-growth upon cooling. For samples S10-S13, which contained an organic amidine catalyst, the solvent was THF. For samples where Tm, Tc, and/or Tg, was determined from the DSC data, these temperatures are included in Table 7. Selected comparisons between samples are described below.

TABLE 7

| Sample | Internal Catalyst | Monomer Additive | Tm | Tc | Tg |
|---|---|---|---|---|---|
| S0 | N/A | N/A | 240 | 134 | 76 |
| S1 | None | None | 228 | 123 | 74 |
| S2 | 2.6 mol % ZnAc₂ | None | 219 | 113 | 67 |
| S3 | 2.6 mol % ZnEH | None | 216 | 106 | 57 |
| S4 | 2.6 mol % ZnAc₂ | 13.2 mol % BHET | | | |
| S5 | 2.6 mol % ZnEH | 13.2 mol % BHET | | | |
| S6 | 2.6 mol % ZnAc₂ | 23.3 mol % CHDM | | | |
| S7 | 2.6 mol % ZnEH | 23.3 mol % CHDM | | | |
| S8 | 2.6 mol % ZnAc₂ | 22.0 mol % BHTD | | | |
| S9 | 2.6 mol % ZnEH | 22.0 mol % BHTD | | | |
| S10 | 0.5 mol % TBD | None | 230 | 126 | |
| S11 | 0.5 mol % DBU | None | 228 | 129 | |
| S12 | 0.5 mol % TBD | 23.3 mol % CHDM | 204 | | 78 |
| S13 | 0.5 mol % DBU | 23.3 mol % CHDM | 202 | | 76 |

S1 exhibited thermal transitions at lower temperatures compared to S0, indicating that melt-processing of PET even in the absence of a catalyst provided some modification of PET structure or morphology, possible including molecular weight reduction. Samples S2 and S3, which included a zinc catalyst during melt-processing, exhibited thermal transitions at lower temperatures than the catalyst-free sample S1, indicating that the internal catalyst provided further molecular weight reduction. Tm, Tc, and Tg for S3, which was melt-processed with zinc ethylhexanoate, were lower than the corresponding temperatures for S2, which was melt-processed with an equimolar amount of zinc acetate. This suggests that zinc ethylhexanoate was slightly more effective than zinc acetate at reducing PET molecular weight during melt-processing.

Samples S4-S9 were melt-processed with a zinc catalyst and a diol monomer additive. From the DSC traces for samples S4-S9, none of S4-S9 exhibited a sharp transition associated with crystallization. In the temperature range where crystallization is expected, the DSC curves of S4-S9 have broad features, unlike the sharp crystallization peaks in the DSC curves of S0-S3, the samples which were not melt-processed with monomer additives. Similarly, the melting transitions of samples S4-S9 generally occurred over a broader temperature range than the melting transitions of samples S1-S3, which were melt-processed without a monomer additive. This broadening of the temperature ranges over which thermal transitions occurred is consistent with amorphization and randomization of the PET structure due to reactions, such as chain scission and transesterification, involving the diol additives.

Each of S4/S5, S6/S7, and S8/S9 is a pair of PET samples that was prepared using the same diol monomer additive but a different zinc catalyst. Comparison of the DSC traces of each shows that within each pair, the sample prepared using zinc ethylhexanoate (S5, S7, S9) exhibited thermal transitions at lower temperatures compared to the corresponding sample that was prepared using an equimolar amount of zinc acetate (S4, S6, S8). This suggests that zinc ethylhexanoate was more effective that zinc acetate at reducing PET molecular weight during melt-processing. As seen in Table 7, this trend is also consistent with results for the S2/S3 pair, which did not contain a diol monomer additive.

The selection of diol monomer additive also affected thermal properties of the melt-processed PET samples. Within each subset of samples that were melt-processed with the same zinc catalyst (i.e., S4/S6/S8 (zinc acetate), and S5/S7/S9 (zinc ethyhexanoate)), the samples that were melt-processed with BHTD had the lowest Tm, the samples that were melt-processed with CHDM had the next highest Tm, and the samples that were melt-processed with BHET had the highest Tm. The melting transitions of the PET samples which were melt-processed with CHDM or BHTD (S6-S9) were less sharp (i.e., occurred over a broader temperature range) compared to the melting transitions of the BHET-containing PET samples (S4 and S5). In particular, Tm's of S4 and S5 were similar to Tm's of samples that were melt-processed without using a diol monomer additive (S1-S3). The similarity of Tm's suggests that incorporating BHET into PET during melt-processing had minimal effect on PET structure, consistent with the high degree of chemical similarity between BHET and PET. The reduced temperatures of thermal transitions for the PET samples that were melt-processed with CHDM or BHTD is indicative of a greater degree of randomization and amorphization of the PET structure in the samples prepared with those monomers.

### Example 8: Organic Catalysts

Samples S10-S13 are PET samples which were melt-processed with 0.5 mol % of an organic catalyst, either 1,5,7-triazabicyclo[4.4.0]dec-5-ene (TBD) or 1,8-diazabicyclo[5.4.0]undec-7-ene (DBU), as the internal catalyst. Table 7 includes Tm and Tc, as determined by DSC, of these samples. Incorporating an organic internal catalyst led to reductions in Tm and Tc compared to non-melt-processed samples. Samples S12 and S13 were melt-processed with an organic internal catalyst and a diol monomer additive. As with samples S4-S9, which were also melt-processed with a monomer additive, the DSC traces of samples S12 and S13 did not exhibit a distinct peak assigned to crystallization. This absence of a sharp crystallization transition is consistent with randomization of the PET structure due to reaction (e.g., transesterification) with the monomer additive.

### Example 9: Depolymerization by Glycolysis

Samples S0 to S9 were subjected to depolymerization by glycolysis in ethylene glycol in the presence of an additional 2 wt % zinc acetate as an external catalyst. To compare depolymerization efficiencies across a range of temperatures, a depolymerization rate was defined as the depolymerization yield after a certain reaction time divided by that reaction time:

$$Depolymerization Rate = \frac{[ \frac{Weight of depolymerized PET}{Initial PET weight} ]}{Reaction time ( hr )}$$

Table 8 lists depolymerization rates for glycolysis of samples S0-S3 at the stated temperatures. The depolymerization rates of S0 and S1 further demonstrate the effect of melt-processing pretreatment on depolymerization rate. S1, which was melt-processed, exhibited faster depolymerization at 160° C., 180° C., and 200° C. compared to S0, which was not-melt-processed. Without intending to be bound by theory, the melt-processing pre-treatment amorphizes the PET, enabling increased access of reactive solvent and catalyst to PET during depolymerization and resulting in increased reaction rates.

S2 and S3 differ from each other in the identity of internal catalyst (zinc acetate and zinc ethylhexanoate, respectively) that was included during melt-processing. Table 8 indicates that while depolymerization proceeded slowly at 120° C., S3 underwent faster depolymerization compared to S2 at 160° C., 180° C., and 200° C.

TABLE 8

Depolymerization Rate (hr⁻¹)

| Sample | 120° C. | 160° C. | 180° C. | 200° C. |
|---|---|---|---|---|
| S0 | | 0.040 | 0.223 | 1.333 |
| S1 | | 0.107 | 0.327 | 1.622 |
| S2 | 0.003 | 0.187 | 1.282 | 2.500 |
| S3 | 0.003 | 0.203 | 1.389 | 2.703 |

The times needed to fully depolymerize each of samples S2-S9 by glycolysis at 170° C. in ethylene glycol with 2 wt. % zinc acetate added as an external catalyst are shown in Table 9. Depolymerization times generally depended on the choice of monomer additive and were generally independent of the choice of internal catalyst. The exception was samples S6/S7, which contained CHDM as an internal catalyst during melt-processing; S7, which had been melt-processed with zinc ethylhexanoate, depolymerized much more quickly than S6, which had been melt-processed with zinc acetate.

TABLE 9

| | Time to Fully |
|---|---|
| | Depolymerize |
| | (glycolysis in EG, |
| Sample | 170° C., 2 wt. % ZnA) |
| S2 | 96 min |
| S3 | 92 min |
| S4 | 65 min |
| S5 | 70 min |
| S6 | 115 min |
| S7 | 24 min |
| S8 | 41 min |
| S9 | 34 min |

Including an organic internal catalyst and a monomer additive during melt-processing enabled fast depolymerization of the resulting melt-processed PET. Table 10 shows the times required to fully depolymerize samples S10-S13 in ethylene glycol at 190° C. No external catalyst was added. PET that had been melt-processed with a combination of organic catalyst and a diol monomer additive was fully depolymerized in only ˜10% of the time needed to fully depolymerize PET that had been melt-processed without a diol monomer additive. After depolymerization, the organic catalyst could be recovered (e.g., via distillation) from the reaction medium and re-used as an internal catalyst in a subsequent melt-processing step with new condensation polymer to be depolymerized.

TABLE 10

| | | | Time to Fully |
|---|---|---|---|
| | | | Depolymerize |
| | Internal | Monomer | (glycolysis in EG, |
| | Catalyst | Additive | 190° C.) |
| S10 | 0.5 mol % TBD | None | 350 min |
| S11 | 0.5 mol % DBU | None | 330 min |
| S12 | 0.5 mol % TBD | 23.3 mol % CHDM | 32 min |
| S13 | 0.5 mol % DBU | 23.3 mol % CHDM | 30 min |

Table 11 lists 100% depolymerization times at 190° C. in ethylene glycol for PET samples which included 0.5 mol. % of an organic catalyst an external catalyst (comparative) or as an internal catalyst along with melt-processing according to the disclosure (samples S12 and S13 described above). Depolymerization of melt-processed PET using an organic internal catalyst was fully depolymerized in under 40 minutes.

TABLE 11

| | | | Time to Fully |
|---|---|---|---|
| | | | Depolymerize |
| Melt- | Internal | External | (glycolysis in EG, |
| Processed? | Catalyst | Catalyst | 190° C.) |
| No | N/A | 0.5 mol % TBD | 325 min |
| No | N/A | 0.5 mol % DBU | 220 min |
| Yes (S12) | 0.5 mol % TBD | N/A | 32 min |
| Yes (S13) | 0.5 mol % DBU | N/A | 38 min |

The above examples demonstrate that rapid depolymerization of PET can be achieved without the use of a metal catalyst, using only organic catalysts which can be removed from the depolymerized reaction mixture via distillation or other suitable means.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In case of conflict, the present description, including definitions, will control.

Throughout the specification, where the compositions, processes, kits, or apparatus are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

What is claimed is:

1. A method for chemically recycling a condensation polymer, the method comprising:
melt-processing a mixture comprising a condensation polymer and a catalyst, thereby forming an amorphous feed material comprising an amorphized condensation polymer and the catalyst, wherein the amorphous feed material has a crystalline polymer content of 30 wt. % or less; and
depolymerizing the amorphous feed material in a reaction medium comprising a reactive solvent, thereby forming a product mixture comprising monomers corresponding to the amorphized condensation polymer.

2. The method of claim 1, wherein the condensation polymer is selected from the group consisting of polyesters, polyamides, and combinations thereof.

3. The method of claim 1, wherein the condensation polymer comprises at least one polyester selected from the group consisting of polyethylene terephthalate (PET), polyethylene terephthalate glycol-modified (PETG) polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polybutylene succinate terephthalate (PBST), polyethylene succinate (PES), poly(butylene succinate/terephthalate/isophthalate)-co-(lactate) (PBSTIL), liquid crystalline polyesters, and combinations thereof.

4. The method of claim 1, wherein the condensation polymer comprises polyethylene terephthalate (PET) and polyethylene terephthalate glycol-modified (PETG).

5. The method of claim 4, wherein the condensation polymer further comprises polylactic acid (PLA).

6. The method of claim 1, wherein the catalyst is selected from the group consisting of metal alkanoates, metal benzoates, metal carbonates, metal sulfates, acids, bases, and combinations thereof.

7. The method of claim 1, wherein the catalyst comprises an organic base.

8. The method of claim 7, wherein the organic base is metal-free.

9. The method of claim 7, wherein the organic base comprises an amidine compound selected from the group consisting of 1,5,7-triazabicyclo[4.4.0]dec-5-ene (TBD) and 1,8-diazabicyclo[5.4.0]undec-7-ene (DBU).

10. The method of claim 7, the organic base has a boiling point in a range of 80° C. to 300° C.

11. The method of claim 7, further comprising:
recovering the organic base from the product mixture; and
optionally performing a subsequent melt-processing of a new condensation polymer with the recovered organic base as the catalyst.

12. The method of claim 1, wherein the catalyst is present in the melt-processed mixture in an amount in a range from 0.1 wt. % to 20 wt. % relative to the melt-processed mixture.

13. The method of claim 1, wherein the catalyst is heat-stable at temperatures experienced during the melt-processing.

14. The method of claim 1, wherein the melt-processed mixture further comprises a monomer reactive with the condensation polymer.

15. The method of claim 14, wherein the monomer is selected from the group consisting of 1,4-cyclohexane dimethanol (CHDM), bis(hydroxymethyl)tricyclo[5.2.1.0^{2.6}]decane (BHTD), and bis(2-hydroxyethyl) terephthalate (BHET).

16. The method of claim 14, wherein the monomer is present in the melt-processed mixture in an amount in a range from 0.5 wt. % to 90 wt. % relative to the melt-processed mixture.

17. The method of claim 1, wherein:
the condensation polymer in the melt-processing mixture has a crystalline polymer content of 20 wt. % or more; and
the amorphized condensation polymer in the amorphous feed material has a crystalline polymer content of 15 wt. % or less.

18. The method of claim 1, wherein melt-processing comprises heating the mixture to a temperature in a range of 260° C. to 320° C.

19. The method of claim 1, further comprising:
quenching the melt-processed amorphous feed material in a liquid medium prior to depolymerizing.

20. The method of claim 1, wherein the reactive solvent is selected from the group consisting of water, mono-alcohols, diols, mono-amines, diamines, and combinations thereof.

21. The method of claim 1, wherein:
the reactive solvent comprises water;
the condensation polymer comprises a polyester; and
the monomers in the product mixture comprise diacids and diols formed from the repeat units of the polyester.

22. The method of claim 1, wherein:
the reactive solvent comprises methanol;
the condensation polymer comprises a polyester; and
the monomers in the product mixture comprise diesters and diols formed from the repeat units of the polyester.

23. The method of claim 1, wherein:
the reactive solvent comprises ethylene glycol;
the condensation polymer comprises a polyester; and
the monomers in the product mixture comprise diesters and diols formed from the repeat units of the polyester.

24. The method of claim 1, wherein:
the reactive solvent comprises water;
the condensation polymer comprises a polyamide; and
the monomers in the product mixture comprise diacids and diamines formed from the repeat units of the polyamide.

25. The method of claim 1, wherein:
the reactive solvent comprises methanol;
the condensation polymer comprises a polyamide; and
the monomers in the product mixture comprise diesters and diamines formed from the repeat units of the polyamide.

26. The method of claim 1, wherein:
the reactive solvent comprises at least one of a mono-amine and a diamine;
the condensation polymer comprises a polyamide; and
the monomers in the product mixture comprise diamides and diamines formed from the repeat units of the polyamide.

27. The method of claim 1, wherein the reaction medium further comprises an external catalyst.

28. The method of claim 19, wherein the external catalyst is present in the reaction medium in an amount in a range from 0.5 wt. % to 10 wt. % relative to the reaction medium

29. The method of claim 1, wherein the reaction medium is free from external catalysts.

30. The method of claim 1, wherein the reaction medium further comprises a surfactant.

31. The method of claim 1, further comprising:
swelling the amorphized condensation polymer with at least one of a gaseous swelling agent and a non-protic solvent swelling agent.

32. The method of claim 1, wherein the reaction medium further comprises at least one of polystyrene (PS) sulfonic acid beads and a hydroxylated pyridine.

33. The method of claim 1, wherein depolymerizing comprises heating the amorphous feed material in the reaction medium to a temperature in a range of 120° C. to 200° C.

34. The method of claim 1, wherein:
the condensation polymer comprises at least one of polyethylene terephthalate (PET) and polyethylene terephthalate glycol-modified (PETG);
the reactive solvent comprises methanol;
the monomers in the product mixture comprise dimethyl terephthalate (DMT), ethylene glycol (EG), and optionally 1,4-cyclohexane dimethanol (CHDM); and
the method further comprises hydrogenating the DMT to form (further) CHDM.

35. The method of claim 1, wherein:
the condensation polymer comprises at least one of polyethylene terephthalate (PET) and polyethylene terephthalate glycol-modified (PETG);
the reactive solvent comprises ethylene glycol; and
the monomers in the product mixture comprise bis(hydroxyethyl)terephthalate.

36. A method for chemically recycling a condensation polymer, the method comprising:
melt-processing a mixture comprising a condensation polymer and, optionally, an internal catalyst, thereby forming an amorphous feed material comprising an amorphized condensation polymer and the internal catalyst (when present), wherein the amorphous feed material has a crystalline polymer content of 30 wt. % or less; and
depolymerizing the amorphous feed material in a reaction medium comprising (i) a reactive solvent and (ii) an external catalyst comprising an organic base, thereby forming a product mixture comprising monomers corresponding to the amorphized condensation polymer.

37. The method of claim 36, wherein the melt-processed mixture does not contain the internal catalyst.

38. The method of claim 36, wherein the melt-processed mixture contains the internal catalyst.

39. The method of claim 36, wherein the melt-processed mixture contains the internal catalyst, and the internal catalyst is other than an organic base.

40. The method of claim 36, wherein the organic base comprises a volatile organic base catalyst.
